(19) United States
(12) Patent Application Publication
(10) Pub. No.: US 2019/0261970 A1
(43) Pub. Date: Aug. 29, 2019

(54) SHAPEABLE ARTICLES WITH STRUCTURED ELEMENTS

(71) Applicant: 3M INNOVATIVE PROPERTIES COMPANY, SAINT PAUL, MN (US)

(72) Inventors: RANJANI V. PARTHASARATHY, WOODBURY, MN (US); HANNAH C. COHEN, SAINT PAUL, MN (US); HAOMING RONG, WOODBURY, MN (US); AMANDA C. ENGLER, WOODBURY, MN (US); WILLIAM BEDINGHAM, WOODBURY, MN (US); NICHOLAS R. ROWLEY, SAINT PAUL, MN (US); KOREY W. KARLS, WOODBURY, MN (US); MICHAEL J. VOSTAL, MINNEAPOLIS, MN (US); MATTHEW T. SCHOLZ, WOODBURY, MN (US); MICHELLE H. STEVENS, BLOOMINGTON, MN (US); CATHERINE D. HEART, NORTH SAINT PAUL, MN (US)

(73) Assignee: 3M INNOVATIVE PROPERTIES COMPANY, SAINT PAUL, MN (US)

(21) Appl. No.: 16/345,812

(22) PCT Filed: Oct. 23, 2017
(86) PCT No.: PCT/US2017/057821

§ 371 Date: Apr. 29, 2019

Related U.S. Application Data
(60) Provisional application No. 62/417,119, filed on Nov. 3, 2016.

(51) Int. Cl. A61B 17/02; A61F 5/01

(52) U.S. Cl. CPC A61B 17/0218; A61B 2017/00849; A61B 2017/0212; A61F 5/01

(57) ABSTRACT

Shapeable articles with structured elements and kits including one or more of the shapeable articles. The shapeable articles described herein have a variety of uses including use as surgical retractors for moving and/or restraining tissue and/or organs to improve access to a surgical site. The shapeable articles described herein can be shaped or manipulated into three-dimensional shapes without the use of tools and hold those shapes after removal of the force required to achieve the shape.

### CROSS REFERENCE TO RELATED APPLICATIONS

This application claims the benefit of U.S. Provisional Patent Application No. 62/417,119, filed Nov. 3, 2016, the disclosure of which is incorporated by reference in its entirety herein.

### FIELD OF THE INVENTION

This invention generally relates to shapeable articles with structured elements, kits incorporating the same and methods of making and/or using the articles.

### BACKGROUND

The restraint of tissues and organs during surgical procedures allows for access and/or visualization of the target tissues and organs that are the focus of the surgical procedures. Although rigid retraction devices and apparatus may be used in some surgical procedures, e.g., the BOOKWALTER retractor system, such retractors may be the source of damage to retracted tissues and/or organs. The potential damage is significant because, for example, there are approximately 1.4 million open abdominal surgeries every year in the United States. About 8.5% of those surgeries lead to ileus, which is a cessation of bowel function. Ileus results in nausea, vomiting, bloating, pain, extended hospital stays, and $1.46 billion increased cost for common abdominal procedures. Additionally, peritoneal adhesions can be found in up to 93% of patients undergoing intra-abdominal surgery.

Attempts to provide malleable or shapeable pads that can be used to restrain tissues and/or organs during surgical procedures are described in, e.g., US Patent Application Publication No. US 2010/0087713. The approaches described in that document do not, however, fully appreciate the issues or solve the problems associated with tissues/organ restraint during surgical procedures.

### SUMMARY

The present invention is directed to shapeable articles with structured elements, kits including one or more of the shapeable articles, and methods of making and/or using the shapeable articles. Although the shapeable articles described herein could have a variety of uses, one use for which they may be well-suited is as surgical retractors used in surgery to move and/or restrain non-target tissue and/or organs to improve access to the target tissue and/or organs.

The shapeable articles described herein can be shaped or manipulated into three-dimensional shapes without the use of tools and hold those shapes after removal of the force required to achieve the shape. In other words, “shapeable” (and variations thereof) as used herein means that an article or component may be plastically deformed from an original shape such that the article or component takes and maintains a selected shape after forming and, further, the shapeable article or component can be further manipulated to return to a configuration that is the same or nearly the same as its original shape. In one or more embodiments, the shapeable articles described herein incorporate one or more ductile metals to provide the required deformation and shapeability.

When used in surgical procedures, the shapeable articles may be used alone or in conjunction with other retraction apparatus (e.g., table-mounted retraction systems such as, e.g., BOOKWALTER retractor systems, etc.). Whether used alone or with other retraction apparatus, the shapeable articles may be manipulated to take a desired shape to assist in the restraint of tissues and/or organs during a surgical procedure.

The shapeable articles may provide one or more advantages when used in surgical procedures. For example in one or more embodiments, the shapeable articles described herein may preferably improve the uniformity of pressure distribution over the surface of the shapeable article, limit or prevent tissue impingement (e.g., “pinching,” etc.), limit unnecessary tissue/organ movement such as tissues/organs slipping out from behind a shapeable article/retractor, and/or reduce the occurrence of pressure points —any one of which may cause tissue or organ damage during a surgical procedure. For example, excessive pressure on tissue can cause damage to that tissue. One manner in which one or more embodiments of the shapeable articles described herein may reduce pressure points is by limiting or even, in one or more embodiments, preventing the formation of creases caused by manipulation of the shapeable article.

Control over bending of the shapeable members of one or more embodiments of shapeable articles as described herein can play a role in reducing pressure points that may be exerted on tissue/organs by the shapeable articles described herein. In particular, creases can be expected to form pressure points (i.e., local areas of increased pressure) when the shapeable articles described herein contact tissue/organs. As a result, reducing the likelihood and/or prominence of creases can play a role in reducing the likelihood of pressure points when using the shapeable articles described herein to restrain tissues/organs.

In one or more embodiments, the shapeable members may bend along lines that follow paths that extend between structured elements of the shapeable members (e.g., land portions as described in connection with one or more embodiments of the shapeable articles described herein). Further, the structured elements may, in one or more embodiments, control or limit the radius of curvature which may also limit creasing of the shapeable members of shapeable articles as described herein.

That same control over bending paths during deformation from a flat to a non-flat configuration may also, in one or more embodiments, control bending of the shapeable members of shapeable articles as described herein when the shapeable articles are being manipulated in attempt to return to their flat configuration before being bent. In other words, control over the bending of a shapeable article from a flat configuration to a bent configuration may also control bending of the shapeable article when being manipulated to return to a flat (or near-flat) configuration after having been bent. That control over bending may, in one or more embodiments, reduce creasing of the shapeable articles. Reduced creasing may, as discussed herein, reduce pressure points and resulting tissue/organ injury when using the shapeable articles described herein to restrain tissue/organs.

Another potential advantage of one or more embodiments of the shapeable articles, when adapted for use in surgical procedures as described herein, may be found in a reduction in surface abrasion of tissues and/or organs that come into contact with the shapeable articles. In one or more embodiments of the shapeable articles as described herein, the outer surfaces of the shapeable articles may have a coefficient of friction within a selected range that is high enough (even when wet) to assist in retention of tissues and/or organs, but is low enough such that excessive abrasion of tissues and organs is significantly reduced during use of the shapeable articles.

In a first aspect, one or more embodiments of the shapeable articles as described herein may include a shapeable member comprising: a malleable core comprising a first major surface and a second major surface, wherein the first major surface and the second major surface are on opposite sides of the malleable core; and a plurality of first structured elements attached to the first major surface of the malleable core, wherein each first structured element of the plurality of first structured elements comprises a protrusion extending away from the malleable core, wherein the plurality of first structured elements are spaced apart from each other by an inter-element distance when the malleable core is in a flat configuration, and wherein the inter-element distance between the plurality of first structured elements changes when the malleable core is deformed into a non-flat configuration. In one or more embodiments, the shapeable member may optionally comprise a plurality of second structured elements attached to the second major surface of the malleable core, wherein each second structured element of the plurality of second structured elements comprises a protrusion extending away from the malleable core, wherein the plurality of second structured elements are spaced apart from each other by an inter-element distance when the malleable core is in a flat configuration, and wherein the inter-element distance between the plurality of second structured elements changes when the malleable core is deformed into a non-flat configuration. The shapeable article also includes a first coversheet attached to the shapeable member, wherein the first coversheet covers the plurality of first structured elements of the shapeable member; and a second coversheet attached to the shapeable member, wherein the second coversheet covers (if present) the plurality of second structured elements of the shapeable member, such that the malleable core is located between first and second coversheets.

In a second aspect, one or more embodiments of a manually shapeable surgical retractor as described herein comprises: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the surgical retractor comprises a perimeter in the general shape of a rectangle, with one or more tabs extending outward from at least one side of the rectangular, wherein each tab of the one or more tabs occupies less than all of the side from which it extends. In one or more embodiments, the shapeable member extends into the area defined the one or more tabs.

In a third aspect, one or more embodiments of a manually shapeable surgical retractor as described herein comprises: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the surgical retractor comprises non-rectangular shape comprising a central portion and two or more fingers extending outwardly from the central portion, wherein the shapeable member extends into each finger of the two or more fingers.

In a fourth aspect, one or more embodiments of a manually shapeable surgical retractor as described herein comprises: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the first coversheet and the second coversheet define a surgical retractor perimeter having a retractor shape, and wherein the shapeable member located between the first coversheet and the second coversheet comprises a member perimeter defining a member shape that is different than the retractor shape.

In a fifth aspect, one or more embodiments of a manually shapeable surgical retractor as described herein comprises: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the first coversheet and the second coversheet define a surgical retractor perimeter having a retractor shape, and wherein the shapeable member located between the first coversheet and the second coversheet comprises a member perimeter defining a member shape that is the same as the retractor shape.

In a sixth aspect, one or more embodiments of a manually shapeable surgical retractor as described herein comprises: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the first coversheet and the second coversheet define an article perimeter having a generally rectangular shape, and wherein the shapeable member located between the first coversheet and the second coversheet comprises a member perimeter defining a non-rectangular member shape that is different than the generally rectangular shape of the article perimeter. In one or more embodiments, the non-rectangular member shape comprises a generally rectangular shape comprising one or more tabs extending outward from at least one side of the rectangular, wherein each tab of the one or more tabs occupies less than all of the side from which it extends.

The above summary is not intended to describe each embodiment or every implementation of the articles, kits, and/or methods as described herein. Rather, a more complete understanding of the invention will become apparent and appreciated by reference to the following Detailed Description and claims in view of the accompanying figures of the drawing.

### BRIEF DESCRIPTIONS OF THE DRAWING

FIG. 1 is a perspective view of one illustrative embodiment of a shapeable article as described herein.

FIG. 2 is an exploded diagram depicting various components that may be found in one illustrative embodiment of a shapeable article as described herein.

FIG. 3 is an enlarged edge view of one illustrative embodiment of a shapeable member that may be used in one or more embodiments of a shapeable article as described herein.

FIG. 4 is a plan view of the shapeable member depicted in FIG. 3.

FIG. 5 is an enlarged cross-sectional view of a portion of one illustrative embodiment of a shapeable member that may be used in one or more embodiments of a shapeable article as described herein.

FIG. 6 is an enlarged diagram depicting one illustrative shapeable member (in a flat configuration) that may be used in one or more embodiments of the shapeable articles described herein.

FIG. 7 depicts the structure of FIG. 6 after manipulation from a flat configuration to a non-planar configuration.

FIG. 8 is an exploded diagram of components that may be used in one or more embodiments of a coversheet on a shapeable article as described herein.

FIG. 9 depicts the shapeable member of FIG. 6 with a coversheet attached to one major surface of the shapeable member.

FIGS. 10-11 depict the shapeable member of FIG. 9 after manipulation from a flat configuration to a non-planar configuration.

FIG. 12A is an enlarged diagram depicting another illustrative embodiment of a shapeable article as described herein.

FIG. 12B is an enlarged cross-sectional view of an optional edge protector that may be incorporated into one or more embodiments of the shapeable articles as described herein.

FIGS. 13-16 depict alternative illustrative embodiments of shapeable members that may be used in one or more embodiments of shapeable articles as described herein.

FIG. 17 depicts another illustrative embodiment of a shapeable article including a window opening formed in an interior of the shapeable member.

FIG. 18 is a cross-sectional view of the shapeable article of FIG. 23 taken along line 18-18 in FIG. 17.

FIG. 19 depicts another illustrative embodiment of a shapeable article including a window opening and an access slit.

FIG. 20 is a cross-sectional view of the shapeable article of FIG. 24 taken along line 20-20 in FIG. 19.

FIG. 21 depicts another illustrative embodiment of a shapeable article as described herein.

FIG. 22 depicts another illustrative embodiment of a shapeable article including tabs as described herein.

FIG. 23 depicts the illustrative embodiment of the shapeable article depicted in FIG. 22 attached to a structure such as a retractor plate.

FIG. 24 depicts another illustrative embodiment of a shapeable article including tabs as described herein.

FIG. 25 depicts another illustrative embodiment of a shapeable article including slits as described herein.

FIG. 26 depicts another illustrative embodiment of a shapeable article including two separate shapeable members and a line of separation in the coversheets located between the shapeable members as described herein.

FIGS. 27-28 depict illustrative embodiments of shapeable articles including one or more folds as described herein.

FIG. 29 depicts illustrative embodiments of shapeable articles including tabs as described herein.

FIG. 30 is a perspective view of the shapeable articles of FIG. 29 arranged in an interlocking relationship.

FIG. 31 depicts illustrative embodiments of shapeable articles including tabs as described herein.

FIG. 32 is a perspective view of the shapeable articles of FIG. 31 arranged in an interlocking relationship.

FIG. 33 depicts illustrative embodiments of shapeable articles including tabs as described herein.

FIG. 34 is a perspective view of the shapeable articles of FIG. 33 arranged in an interlocking relationship.

FIG. 35 depicts another illustrative embodiment of a shapeable article including tabs as described herein.

FIG. 36 is a perspective view of the shapeable article of FIG. 35 arranged in an interlocking relationship.

FIG. 37 is a schematic diagram of one illustrative embodiment of a kit that may include one or more shapeable articles as described herein along with other items in a sealed package.

### DETAILED DESCRIPTION

In the following description, reference is made to the accompanying figures of the drawing which form a part hereof, and in which are shown, by way of illustration, specific embodiments. It is to be understood that other embodiments may be utilized and changes may be made without departing from the scope of the present invention.

Although described in terms of their use in surgical procedures, the shapeable articles described herein may find many uses in other applications as well, and description of the illustrative embodiments in a surgical setting should not be construed as limiting the use of shapeable articles constructed according to the principles described herein to surgical procedures.

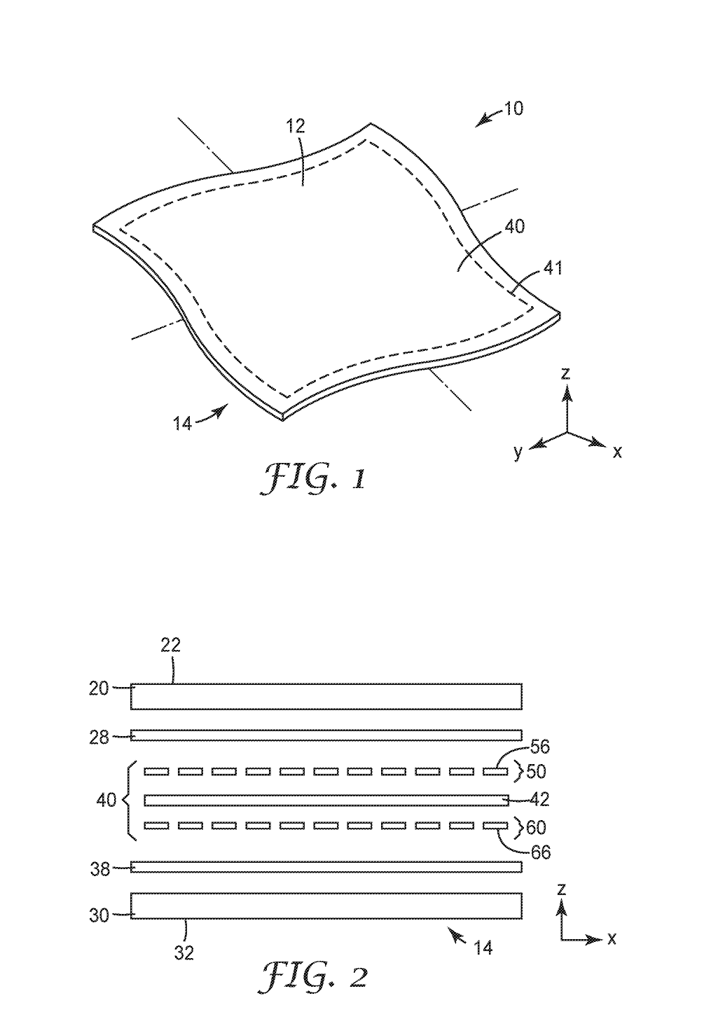

One illustrative embodiment of a shapeable article 10 as described herein is depicted in FIG. 1. The shapeable article 10 includes two major surfaces 12 and 14 located on opposite sides of the shapeable article 10 which is provided in the form of a sheet or pad. Although the shapeable article 10 can take a flat configuration in which the two major surfaces 12 and 14 are essentially planar surfaces, the shapeable articles 10 as described herein can be manipulated into non-planar configurations. In other words, although the shapeable articles 10 can be provided in a configuration in which the surfaces 12 and 14 essentially lie in an X-Y plane with the thickness of the shapeable article 10 extending in a Z direction, the shapeable articles 10 can be manipulated such that the first and second major surfaces 12 and 14 no longer lie in a plane, e.g., are curved or otherwise non-planar. In one or more embodiments, however, the manipulation does not substantially change the thickness of the shapeable article 10 between its first and second major surfaces 12 and 14. Rather, the shapeable articles 10 form complementary curves on major surfaces 12 and 14.

The shapeable article 10 in FIG. 1 is depicted after manipulation from a flat planar configuration such that first major surface 12 exhibits a combination of convex and concave surfaces while the second major surface 14 also exhibits a combination of convex and concave surfaces that are the opposite of those found on first major surface 12. In other words, where first major surface 12 exhibits a convex surface, second major surface 14 exhibits a complementary concave surface and vice versa. The X, Y and Z axes of a representative Cartesian coordinate system depicted in FIG. 1 may be included in figures where helpful to provide a frame of reference when describing the shapeable articles and methods described herein.

As discussed above, the shapeable articles described herein can be shaped or manipulated into three-dimensional shapes without the use of tools and hold those shapes after removal of the force required to achieve the shape. In those embodiments in which the shapeable articles are to be manually formed into a desired shape or configuration, it may be beneficial to limit the force needed to bend or form the shapeable articles. In one or more embodiments, the shapeable articles as described herein may be defined in terms of stiffness. In particular, in one or more embodiments, the shapeable articles described herein may have a stiffness allowing for manual deformation of the shapeable articles into desired shapes without requiring tools to do so. In one or more embodiments, the shapeable articles as described herein may have a stiffness of 100 N or less, 80 N or less, 60 N or less, 40 N or less, 20 N or less, or even 10 N or less as measured using a Three-Point Bend test. In other words, bending of the shapeable article 10 around either of the axes depicted in FIG. 1 may, in one or more embodiments, require a force of 100 N or less, 80 N or less, 60 N or less, 40 N or less, 20 N or less, or even 10 N or less according to the identified test method.

In addition to being manually deformable into one or more selected shapes as described herein, one or more embodiments of the shapeable articles described herein exhibit limited elastic recovery after being deformed. For example, in one or more embodiments, the shapeable articles described herein recover 20% or less (or in one or more alternative embodiments, 10% or less) of any deflection imparted to them during deformation after a period of 5 minutes or less (or, in one or more embodiments, 2 minutes or less) after removal of the force applied to deform the shapeable article. In one or more embodiments of the shapeable articles described herein, the shapeable articles may exhibit little or no elastic recovery (e.g., have an elastic recovery of 0% or more) of any deflection imparted to them during deformation within the time periods described above.

The shapeable articles described herein are constructed of multiple components attached together to form a shapeable articles such as shapeable article 10 depicted in FIG. 1. One illustrative embodiment of components that may be included in shapeable articles as described herein are depicted in the exploded diagram of FIG. 2. The depicted shapeable article 10 includes a first coversheet 20, second coversheet 30, and shapeable member 40 attached together to form the shapeable article 10. The first coversheet 20 can be attached to a first major surface of the shapeable member 40 while the second coversheet 30 is attached to a second major surface of the shapeable member 40. The first and second major surfaces of the shapeable member 40 are, like the major surfaces 12 and 14 of the shapeable article 10, located on opposite sides of the shapeable member 40.

In one or more embodiments, the first coversheet 20 may be attached to the shapeable member 40 and/or to the second coversheet 30 on the opposite side of the shapeable member 40 using adhesive 28. Similarly, the second coversheet 30 may, in one or more embodiments, may be attached to the shapeable member 40 and/or to the first coversheet 20 using adhesive 38. In one or more embodiments, the first coversheet 20 may be described as being attached to the second coversheet 30 about a perimeter of the shapeable member 40. As seen in, e.g., FIG. 1, the perimeter 41 of the shapeable member 40 is inset from the perimeter of the coversheets and, in such an embodiment, the first and second coversheets 20 and 30 may be attached to each other about the perimeter of the shapeable member 40. The shapeable member 40 itself may be attached to the first and second coversheets 20 and 30 using any suitable technique, e.g., adhesives, welding (one or more of thermal, chemical, and mechanical welding), sewing, mechanical fasteners (e.g., hook and loop fasteners, stem fasteners (e.g., 3M DUAL LOCK reclosable stem fasteners), etc.), riveting, stitching, crimping, etc. over any necessary portion of the perimeter to maintain the components in position with each other.

Further, although adhesives 28 and 38 are depicted as being continuous layers in FIG. 2, the adhesives 28 and 38 used to attach coversheets 20 and 30 to each other and/or to a shapeable member 40 may be continuous or discontinuous (e.g., pattern coated, etc.). One or both of the adhesives 28 and 38 may, in one or more embodiments, be a pressure sensitive adhesive such as, e.g., an acrylate, polyurethane, polyolefin, styrene copolymer or a combination thereof; a hot melt adhesive such as a polyolefin or modified polyolefin (ethylene vinylacetate, ethylene acrylates such as ethylene methylacrylate, acrylates such as KURARITY (from Kuraray) and the like), or a curable adhesive such a 2 part silicone, epoxy, or polyurethane.

Although adhesives are used in constructing one or more embodiments of the shapeable articles 10 as described herein, coversheets may be attached to each other and/or the shapeable member 40 of a shapeable article 10 as described herein through the use of other techniques such as, e.g., insert molding, etc. If insert molding is used, the first and second coversheets may not be separately discernible layers/components around the perimeter of the shapeable member 40 because the material used for the coversheets may form a contiguous mass about the perimeter 41 of the shapeable member 40. In still other alternative embodiments, the first and second coversheets may be attached to each other about the perimeter 41 of the shapeable member 40 by any suitable technique, e.g., adhesives, welding (one or more of thermal, chemical, and mechanical welding), sewing, mechanical fasteners (e.g., hook and loop fasteners, stem fasteners (e.g., 3M DUAL LOCK reclosable stem fasteners), etc.), riveting, stitching, crimping, etc.

The first coversheet 20 and second coversheet 30 on a shapeable article as described herein may be constructed of the same or different components depending on the intended use of the shapeable articles described herein. When intended for use in surgical procedures, the first and second coversheets 20 and 30 may include a variety of different components configured to assist in restraining tissue and/or organs while reducing trauma due to, e.g., pressure points, abrasion, etc.

In one or more embodiments, the coversheets used in shapeable articles described herein may include one or more components such as, e.g., foam layers, polymeric films/sheets, nonwoven sheets, woven sheets, knitted sheets, mesh sheets, net sheets, etc. Further, a coversheet as used in connection with the shapeable articles described herein may include two or more different layers of the same material, e.g., a single coversheet may include two foam layers, two polymeric film/sheet layers, etc. Further, one or more layers of any coversheet used in one or more embodiments of the shapeable articles as described herein may be absorbent such that the layer or layers absorb water, normal saline, etc. before or during use as desired. Still further, one or more layers of any coversheet used in one or more embodiments of the shapeable articles as described herein may be non-absorbent such that the layer or layers do not absorb water, normal saline, etc. before or during use as desired.

In one or more embodiments in which a coversheet used on a shapeable article as described herein includes a compressible layer to, e.g., control the formation of pressure points on tissue and/or organs that come into contact with the shapeable article. Examples of some potentially suitable compressible layers that may be used in connection with one or more embodiments of the shapeable articles described herein may include open or closed cell foams, silicone sheets, polyurethanes, silicone polyureas, silicones, cotton, polyesters, ethylene vinyl acetate, etc.

In one or more embodiments, a compressible coversheet used in a shapeable article as described herein may exhibit compression set of 50% or less (or, alternatively, 30% or less, or even 20% or less) of an original thickness when tested according to ASTM D3575. In one or more embodiments, a compressible coversheet component used in a shapeable article as described herein may be in the form of a sheet of closed cell EVA copolymer foam, one potentially suitable version of which is available from Sekisui Voltek, Lawrence Mass., USA under the designation VOLARA Type EO with a density of 3.2 kilograms per cubic meter (e.g., 2 pounds per cubic foot) and a thickness of 1.57 millimeters (e.g., 0.062 inches).

In one or more embodiments of the shapeable articles described herein, one or both of the coversheets attached to a shapeable member may be extensible, i.e., may exhibit some extensibility in response to tension forces applied in a cross sheet direction (where a cross sheet direction correspond generally to directions lying in a plane occupied by the coversheet when the coversheet is in a flat configuration). A coversheet used in one or more embodiments of the shapeable articles described herein may be described as exhibiting tensile elongation (without tearing, ripping, etc.) of 10% or more, 20% or more, or 30% or more at a lower end in response to tensile forces applied in a cross sheet direction according to ASTM D5034 Grab tensile test. At an upper end, the coversheets used in one or more embodiments of the shapeable articles described herein may be described as exhibiting tensile elongation (up to failure (e.g., fracture, tearing, etc.)) of 1000% or less, 500% or less, 250% or less, 200% or less, 150% or less, 120% or less, or 110% or less according to ASTM D5034 Grab tensile test.

The extensibility of a coversheet used in one or more embodiments of shapeable articles as described herein may be elastic extensibility, where elongation of the coversheet may be substantially recovered after removal of any tensile forces causing the elongation. In one or more embodiments, one or both the coversheets on a shapeable article as described herein may recover 30% or more, 40% or more, or 50% or more of any elongation within 60 seconds or less of removal of any tensile forces causing elongation of a coversheet.

In one or more embodiments of the shapeable articles described herein, one or both of the coversheets 20 and 30 may include an outer surface 22 or 32, respectively, facing away from the shapeable member with a coefficient of friction that is, as described herein, high enough to assist in retention of tissues and/or organs, but also low enough such that excessive abrasion of tissues and organs does not occur during use of the shapeable articles in surgical procedures. Such frictional properties are also, in one or more embodiments, found in shapeable articles that are fully hydrated with, e.g., water, normal saline (0.90% wt/wt sodium chloride in water), etc. In one or more embodiments, the outer surfaces of coversheets used in one or more embodiments of a shapeable member as described herein may exhibit a mean coefficient of friction of at least 0.2. At an upper limit, it may be beneficial to provide a shapeable article as described herein that includes an outer surface having a mean coefficient of friction of up to 0.45. In still other alternative embodiments, the outer surface of a shapeable article as described herein may exhibit a mean coefficient of friction of up to only 0.35.

The frictional properties of the outer surfaces of coversheets used in one or more embodiments of shapeable articles as described herein may be controlled using materials that are either used to construct the outer surface and/or coated on the outer surface. Some potentially suitable examples of materials and constructions that may provide desirable coefficients of friction are the materials containing silicone polyurea copolymers described in, e.g., U.S. Provisional Patent Application No. 62/417,146, filed Nov. 3, 2016 and titled SILICONE COPOLYMERS, METHODS OF MAKING, AND ARTICLES.

One illustrative embodiment of components that may be found in a shapeable member 40 as provided in the shapeable article 10 is depicted in FIG. 2. The shapeable member 40 includes a malleable core 42 having first and second major surfaces on opposite sides of the malleable core 42. The shapeable member 40 further includes first structured elements 50 (individually designated by ref. no. 56) attached to the first major surface of the malleable core 42. Each first structured element 56 forms a protrusion extending away from the malleable core 42. The first structured elements 56 are spaced apart from each other by an inter-element distance when the malleable core 42 is in a flat configuration (as depicted in FIG. 2). As discussed herein, the inter-element distance between the first structured elements 56 changes when the malleable core 42 is deformed into a non-flat configuration.

The shapeable member 40 further includes second structured elements 60 (individually designated by ref. no. 66) attached to the second major surface of the malleable core 42. Each second structured element 66 forms a protrusion extending away from the malleable core 42. The second structured elements 66 are also spaced apart from each other by an inter-element distance when the malleable core 42 is in a flat configuration (as depicted in FIG. 2). As discussed herein, the inter-element distance between the second structured elements 66 changes when the malleable core 42 is deformed into a non-flat configuration. The structured elements 56 of group 50 and the structured elements 66 of group 60 may be attached to the major surfaces of malleable core 42 using one or more techniques such as, e.g., adhesives, welding (one or more of thermal, chemical, and mechanical welding), sewing, mechanical fasteners (e.g., hook and loop fasteners, stem fasteners (e.g., 3M DUAL LOCK reclosable stem fasteners), etc.), riveting, stitching, crimping, etc. If an adhesive is used, it may, in one or more embodiments, be a pressure sensitive adhesive such as, e.g., an acrylate, polyurethane, polyolefin, styrene copolymer or a combination thereof; a hot melt adhesive such as a polyolefin or modified polyolefin (ethylene vinylacetate, ethylene acrylates such as ethylene methylacrylate, acrylates such as KURARITY (from Kuraray) and the like), or a curable adhesive such a 2 part silicone, epoxy, or polyurethane.

Although the illustrative embodiment of shapeable article 10 includes a shapeable member 40 including two sets 50 and 60 of structured elements 56 and 66 on opposite sides of a malleable core 42 as described herein, one or more alternative embodiments of shapeable articles as described herein may include only one set of structured elements on one side of a malleable core 42 as described herein.

A single layer of metal foil may be particularly useful to provide malleability/ductility to a malleable core 42 used in a shapeable member as described herein, but in other alternative embodiments, the malleable core 42 may include one or more layers of metal foil, strips of metal foil, metal mesh, metal screens, etc. to provide malleability to the malleable core 42. The malleable core 42 may be constructed of any suitable malleable/ductile metal such as, e.g., aluminum, copper, stainless steel, etc. In one or more embodiments, a ductile metal sheet/foil may be continuous, perforated, or porous. One suitable metal foil may include, e.g., a continuous sheet of 1100 Series aluminum foil with a thickness of, e.g., 0.005 inches (about 0.127 millimeters). In still other embodiments, the malleable core 42 may be constructed of materials that do not include a metal foil to provide malleability, e.g., shape memory polymers, metal/polymeric composites, highly filled polymeric compositions generally having greater than 50%, 60%, and even 70% wt/wt filler (e.g., one or more inorganic fillers, etc.), etc.

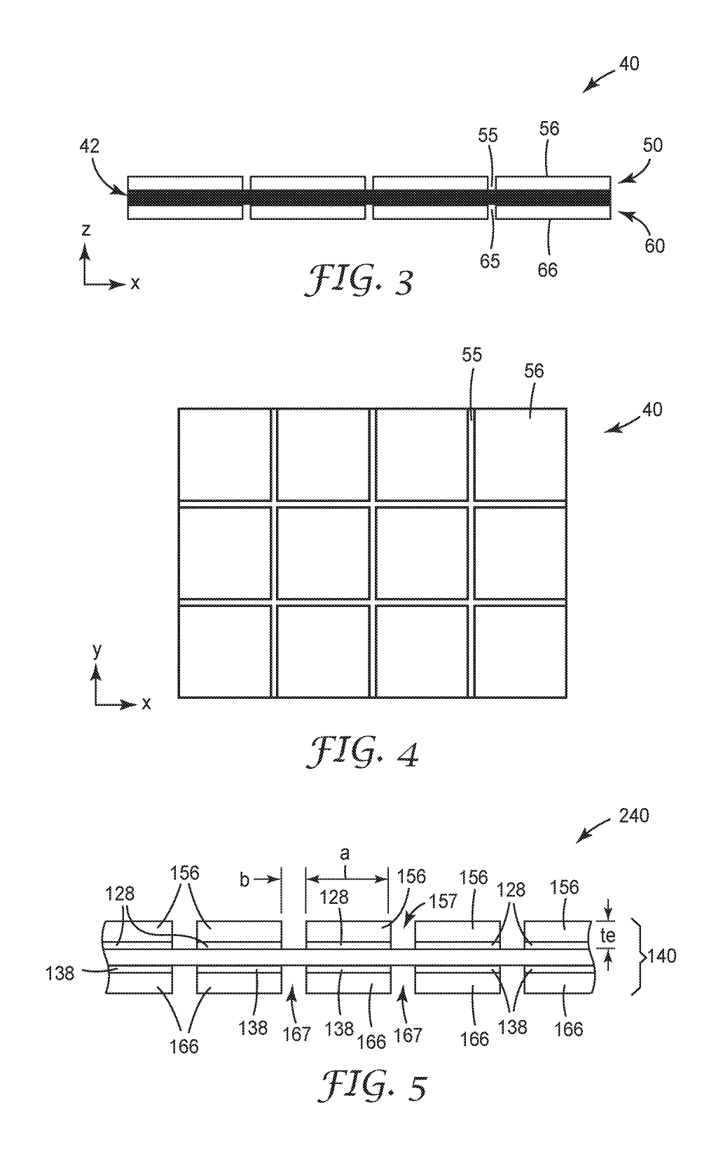

FIG. 3 is an enlarged edge view and FIG. 4 is a plan view of a shapeable member 40 including a first group 50 structured elements 56 on one side of malleable core 42 and a second group 60 of structured elements 66 on the second major surface of malleable core 42. The structured elements 56 on the first side of malleable core 42 are separated from each other by a land portion 55 exposed through the inter-element distance by which the structured elements 56 are spaced apart. Similarly, the structured elements 66 on the second side of malleable core 42 are separated from each other by a land portion 65 exposed through the inter-element distance by which the structured elements 66 are spaced apart.

In the embodiment depicted in FIG. 3, the structured elements 56 on the first major surface of malleable core 42 are aligned with the structured elements 66 on the second surface of malleable core 42, where that alignment is along the Z axis as depicted in FIG. 3. As a result, the land portion 55 between structured elements 56 on the first major surface of malleable core 42 may also, in one or more embodiments, be aligned with the land portions 65 located between the structured elements 66 on the second major surface of malleable core 42.

The inter-element spacing between the structured elements 56 and 66 may, in one or more embodiments, provide structural advantages to the shapeable member when deformed from its flat configuration. In particular, the inter-element spacing between structured elements 56 and 66 may result in bending of the shapeable member 40, when deformed from a flat, planar configuration (see, e.g., FIG. 1) that follows the land portions 55 and 65 located between the structured elements 56 and 66. In other words, in one or more embodiments the structured elements 56 and 66 may not, themselves, typically deform in response to folding or manipulation of a shapeable article including the shapeable member 40. Rather, bending or deformation of the shapeable member preferably occurs in the land portions 55 and 65 between the structured elements 56 and 66.

The control over bending of the shapeable member 40 of one or more embodiments of shapeable articles as described herein may play a role in reducing pressure points that may be exerted on tissue/organs by the shapeable articles described herein. In particular, creases can be expected to form pressure points (i.e., local areas of increased pressure) when the shapeable articles described herein contact tissue/organs. As a result, reducing the likelihood and/or prominence of creases can play a role in reducing the likelihood of pressure points when using the shapeable articles described herein to restrain tissues/organs.

FIG. 5 depicts another illustrative embodiment of a shapeable member 140 which may be used in one or more embodiments of a shapeable article as described herein. The depicted shapeable member 140 includes first structured elements 156 attached to a first major surface of a malleable core 142 and second structured elements 166 attached to a second major surface of malleable core 142. The first structured elements 156 are adhesively attached to the malleable core 142 using adhesive 128 as depicted in FIG. 5 (although other attachment techniques could be used as described herein). Similarly, the second structured elements 166 are adhesively attached to the malleable core 142 using adhesive 138 as depicted in FIG. 5 (although other attachment techniques could be used as described herein). Other features depicted in FIG. 5 include spaces or gaps 157 and 167 between structured elements 156 and 166, respectively.

The structured elements may, in one or more embodiments of shapeable articles as described herein, be characterized in terms of its thickness As seen in the illustrative embodiment depicted in FIG. 5, each structured element 156 has a maximum element thickness (te) measured in the Z direction transverse to the major surfaces of the malleable core 142 (when the malleable core 142 is in a flat configuration as seen in, e.g., FIG. 5). As depicted in, e.g., FIG. 5, one or more embodiments of the shapeable articles as described herein may include structured elements 156 with a maximum element thickness (te) of 2 centimeters or less. In one or more embodiments, the maximum element thickness may be, e.g., 0.5 centimeters or less (e.g., 0.2 centimeters). The structured elements 166 on the second major surface of malleable core 142 may also, in one or more embodiments, have the same dimensions as structured elements 156 although, in one or more alternative embodiments, the structured elements 166 on the second major surface of malleable core 142 may have different dimensions.

In one or more embodiments of shapeable members used in shapeable articles as described herein, the structured elements may also be characterized in terms of the size of the structured elements. As depicted in, e.g., FIG. 5, one or more embodiments of the shapeable articles as described herein may include structured elements 156 with a maximum cross-sheet dimension (a) of 2.5 centimeters or less when the malleable core 142 is in a flat configuration (in one illustrative embodiment, the maximum cross-sheet dimension (a) may be, e.g., 1 centimeter or less). The structured elements 166 on the second major surface of malleable core 142 may also, in one or more embodiments, have the same dimensions as structured elements 156 although, in one or more alternative embodiments, the structured elements 166 on the second major surface of malleable core 142 may have different dimensions.

In one or more embodiments of shapeable members used in shapeable articles as described herein, the structured elements used in the shapeable members may also be described in terms of the inter-element distance between two or more neighboring structured element pairs of a shapeable member. With reference to FIG. 5, the structured elements 156 of two or more neighboring structured element pairs on the first major surface of malleable core 142 may be separated from each other by an inter-element distance (b) when the major surfaces of the malleable core 142 are in a flat configuration (as seen in, e.g., FIG. 5). The structured elements 166 on the second major surface of malleable core 142 may also, in one or more embodiments, be spaced apart from each other by an inter-element distance having the same dimensions as structured elements 156 although, in one or more alternative embodiments, the structured elements 166 on the second major surface of malleable core 142 may be spaced apart from each other by different dimensions.

In one or more embodiments of shapeable members used in shapeable articles as described herein, the inter-element distance (b) between structured elements of two or more neighboring structured element pairs may be a maximum of 2.5 centimeters or less (in one or more embodiments, the maximum cross-sheet dimension (b) may be, e.g., 0.5 centimeter or less).

In one or more embodiments, the maximum element thickness (te) of the structured elements on a sheet may be equal to or greater than maximum inter-element distance (b).

In one or more embodiments, for a projection of a major surface of the shapeable members used in one or more embodiments of the shapeable articles described herein, the maximum area occupied on that projection by the spaces between the structured elements may be less than the area on that projection that is occupied by the structured elements.

Although the depicted illustrative embodiment of shapeable member 140 includes structured elements 156 and 166 that are separated from each other by an inter-element distances that are uniform when moving away from the malleable core 142, in one or more alternative embodiments, the inter-element distance between neighboring pairs of structured elements on a malleable core may change when moving away from the major surfaces of the malleable core to which the structured elements are attached.

Further, in one or more embodiments, the inter-element distance (b) between the structured elements 156 may increase when moving away from the malleable core 142 while the inter-element distance between the structured elements 166 may decrease when moving away from the malleable core 142 when the shapeable member 140 is deformed such that the first major surface of malleable core 142 forms a concave shape while the opposite major surface of malleable core 142 forms a convex shape.

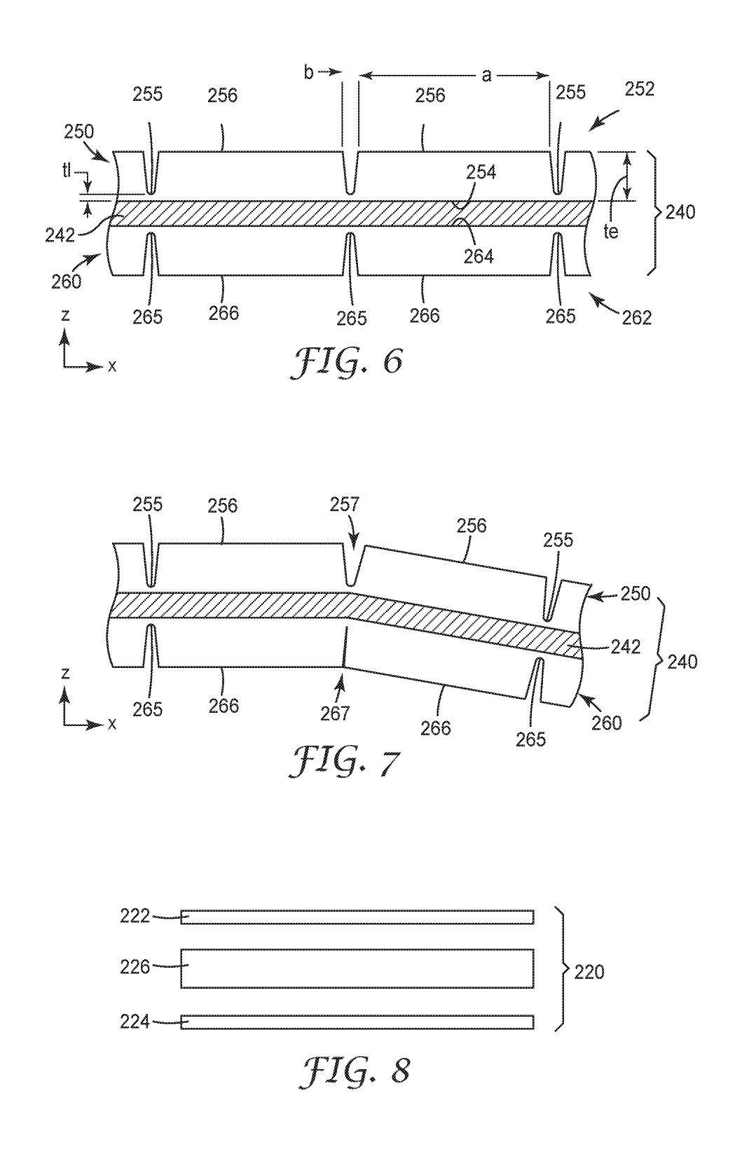

Although one or more embodiments of shapeable members as described herein may include structured elements attached to a malleable core in which the structured elements are not connected to each other but for their connection to the malleable core, in one or more alternative embodiments, the structured elements may be attached to each other as well as to a malleable core. One illustrative embodiment of a shapeable member including structured elements that are attached to one another on at least one side of a malleable core is depicted in FIGS. 6-7. The shapeable member 240 includes first structured elements 256 attached to each other to form a first sheet 250 and second structured elements 266 attached to each other to form second sheet 260. Both the first sheet 250 and the second sheet 260 are attached to a malleable core 242 located between the first sheet 250 and the second sheet 260.

The first sheet 250 includes a land portion 255 that extends between and connects structured elements 256. Each of the structured elements 256 forms a protrusion extending from the land portion 255 of the first sheet 250. Structured elements 256 that are surrounded by other structured elements 256 on the interior of the array are surrounded by the land portion 255, while structured elements 256 located on an edge of the malleable core 240 may include a land portion only about a portion of the perimeter of the structured element 256.

Similarly, the second sheet 260 includes a land portion 265 that extends between and connects structured elements 266 of the second sheet 260. Each of the structured elements 266 forms a protrusion extending from the land portion 265 of the second sheet 260. Structured elements 266 that are surrounded by other structured elements 266 on the interior of the array are surrounded by the land portion 265, while structured elements 266 located on an edge of the malleable core 240 may include a land portion only about a portion of the perimeter of the structured element 266.

In one or more embodiments, all or only a portion of the first sheet 250 is attached to the malleable core 242. For example, in one or more embodiments, only the land portions of the first sheet 250 and/or the second sheet 260 may be attached to the malleable core 242, only the structured elements 256 and/or 266 of the first sheet 250 and/or the second sheet 260 may be attached to the malleable core 242, or only portions of any of the structured elements may be attached to the malleable core 242. In essence, attachment of the first sheet 250 and/or the second sheet 262 the malleable core 242 may be accomplished by any technique or combination of techniques sufficient to provide a unitary shapeable member 240 capable of functioning as described herein, e.g., adhesives, welding, (one or more of thermal, chemical, and mechanical welding), sewing, mechanical fasteners (e.g., hook and loop fasteners, stem fasteners (e.g., 3M DUAL LOCK reclosable stem fasteners), etc.), riveting, stitching, crimping, etc.

In one or more embodiments of shapeable articles as described herein, the structured elements 256 and/or 266 may be provided with cavities or depressions facing the malleable core 242. In other embodiments, the structured elements 256 and/or 256 may be hollow.

The malleable core 242 may consist essentially of a single layer of metal foil in one or more embodiments, but in other alternative embodiments, the malleable core 242 may include one or more layers of metal foil, strips of metal foil, metal mesh, metal screens, etc. to provide malleability to the shapeable member 240. The malleable core 242 may be constructed of any suitable malleable/ductile metal such as, e.g., aluminum, copper, stainless steel, etc. In one or more embodiments, a ductile metal sheet/foil may be continuous, perforated, or porous. One suitable metal foil may include, e.g., a continuous sheet of 1100 Series aluminum foil with a thickness of, e.g., 0.005 inches (about 0.127 millimeters). In still other embodiments, the malleable core 242 may be constructed of materials that do not include a metal foil to provide malleability, e.g., shape memory polymers, metal/polymeric composites, highly filled polymeric compositions generally having greater than 50%, 60%, and even 70% wt/wt filler (e.g., one or more inorganic fillers, etc.), etc.

Each of the structured element may, in one or more embodiments of shapeable articles as described herein, be characterized in terms of its thickness relative to the land portion connecting and extending between the structured elements in shapeable members such as shapeable member 240 depicted in FIGS. 6 and 7. As seen in that illustrative embodiment, each structured element 256 has a maximum element thickness (te) measured in the Z direction transverse to the major surface of the malleable core 242 when the malleable core 242 is in a flat configuration (as seen in, e.g., FIG. 6). Also as seen in FIG. 6, the land portion 255 extending between and connecting structured elements 256 has a land thickness (tl) that is also measured transverse to the major surface of the malleable core 242 when the malleable core 242 is in a flat configuration. In one or more embodiments of shapeable members in shapeable articles as described herein that include structured elements connected by a land portion, the element thickness (te) is greater than the land thickness (tl).

In one or more embodiments, the land portion 255 extending between and connecting structured elements 256 may have a uniform land thickness (tl) over the first sheet 250. In one or more alternative embodiments, however, the land thickness (tl) may vary between different pairs of structured elements. Similarly, the land portion 265 extending between and connecting structured elements 266 on the second sheet may have a uniform land thickness (tl) over the second sheet 260, although in one or more alternative embodiments, the land thickness (tl) may vary over the second sheet 260.

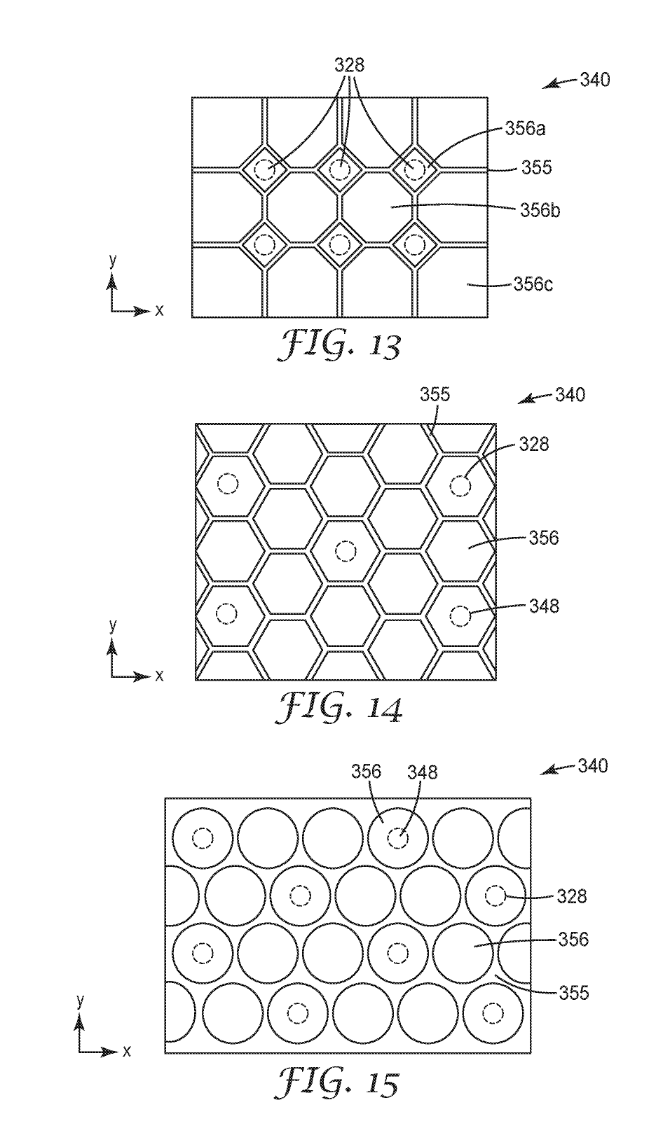

In one or more embodiments of shapeable articles as described herein, the structured elements connected by a land portion may be characterized in terms of the size of the structured elements. As depicted in, e.g., FIG. 13, one or more embodiments of the shapeable articles as described herein may include structured elements 256 on the first sheet 250 with a maximum cross-sheet dimension (a) of 2.5 centimeters or less when the land portion 255 of the first sheet 250 is in a flat configuration on a malleable core 242 that is also in a flat configuration (in one or more alternative embodiments, the maximum cross-sheet dimension (a) may be 1 centimeter or less). The structured elements 266 on the second sheet 260 may also, in one or more embodiments, have the same dimensions as structured elements 256 on the first sheet 250 although, in one or more alternative embodiments, the structured elements 266 on the second sheet 260 may have different dimensions.

In one or more embodiments of shapeable members used in shapeable articles as described herein, the structured elements used in the shapeable members may also be described in terms of the distance between two or more neighboring structured element pairs. With reference to FIG. 6, the structured elements 256 of two or more neighboring structured element pairs on the first sheet 250 may be separated from each other by an inter-element distance (b) when the land portion 255 of the first sheet 250 is in a flat configuration on a malleable core 242 that is also in a flat configuration (as seen in, e.g., FIG. 6). The separation between structured elements forms spaces or gaps 257 and 267 between structured elements 256 and 266, respectively.

In one or more embodiments such as that depicted in FIG. 6, the inter-element distance (b) increases when moving away from the land portion 255 (or the malleable core 242). In one or more alternative embodiments, however, the inter-element distance be may be uniform (i.e., substantially unchanging) when moving away from the land portion 255 of the first sheet 250 (or the malleable core 242). The structured elements 266 on the second sheet 260 may also be similarly separated from each other.

In one or more embodiments of shapeable members used in shapeable articles as described herein, the inter-element distance (b) between structured elements of two or more neighboring structured element pairs may be a maximum of 2 centimeters or less (in one or more embodiments, the maximum inter-element distance (b) may be, e.g., 0.5 centimeter or less, 0.2 centimeters or less, etc.).

As seen in FIG. 7, in one or more embodiments the inter-element distance (b) between the structured elements 256 increases when moving away from the malleable core 242 while the inter-element distance between the structured elements 266 decreases when moving away from the malleable core 242 when the shapeable member 240 is deformed such that the first major surface of malleable core 242 forms a concave shape while the opposite major surface of malleable core 242 forms a convex shape. As a result, the space or gap 257 between structured elements 256 increases while the space or gap 267 between structured elements 266 decreases.

Separation of structured elements by an inter-element distance (b) that increases (e.g., as depicted in FIG. 6) when moving away from the malleable core may, in one or more embodiments, enhance the ability of the shapeable member to be deformed such that the shapeable member can form curved configurations with smaller radii of curvature. The ability of the shapeable member to be deformed may be enhanced because of the additional clearance between adjacent structured elements.

Further, control over the amount of curvature that can be provided by a shapeable member may be provided by contact between adjacent neighboring pairs of structured elements. As seen in, e.g., FIG. 7, contact between neighboring pairs of structured elements 266 may limit further deformation of the shapeable member 240.

Although the land portions 255 and 265 of the first and second sheets 250 and 260 depicted in FIGS. 6 and 7 are depicted as constructed of a single material, in one or more embodiments, the structured elements 256 and 266 could be provided as separate and discrete articles attached to a separate and discrete land portion 255 or 265.

The sheets used in one or more embodiments of a shapeable member 240 used in shapeable articles as described herein may be constructed of a variety of different materials. For example, in one or more embodiments, one or both of the sheets 250 and 260 may be provided in the form of a polymeric sheet including both land portions 255 and 265 and structured elements 256 and 266 as described herein. In one or more alternative embodiments, one or both of the sheets 250 and 260 may consist essentially of a polymeric sheet that forms both the land portions and the structured elements of a given sheet used in a shapeable member of shapeable article as described herein.

In general, the materials used in at least the land portions of the sheets 250 and 260 may be flexible such that the shapeable member 240 can be manipulated as seen in, e.g., FIG. 7. In one or more embodiments, the sheets 250 and/or 260 may be constructed of, e.g., one or more polymeric materials that offer sufficient flexibility to the shapeable member 240 when constructed with a malleable core.

Although the cover sheets 20 and 30 on shapeable member 40 as depicted in FIG. 2 include a single layer of material, coversheets used in connection with shapeable members to form shapeable articles as described herein may be constructed of two or more layers of materials. One illustrative embodiment of a coversheet 220 including multiple layers is depicted in FIG. 8. In that depicted illustrative embodiment, coversheet 220 includes a base layer 224, an intermediate layer 226 and an outer covering 222. Although the depicted illustrative embodiment of coversheet 220 includes three layers, it should be understood that coversheet used on shapeable articles as described herein may include only two layers or more than three layers.

The various components of the coversheet 220 may, in one or more embodiments, include one or more layers selected from a foam layer, a polymeric film, a nonwoven sheet, a woven sheet, a knitted sheet, a mesh sheet, a net sheet, etc. as described herein with respect to other illustrative embodiments of coversheets used in shapeable articles as described herein. Any foam layers provided in coversheet 220 may include characteristics described herein with respect to other foam layers in other coversheets used in other illustrative embodiments of shapeable articles as described herein.

In one or more embodiments, the base layer 224 may be formed of an extensible material which, in some instances, may be an elastically extensible material. In one or more embodiments of shapeable articles as described herein, the base layer 224 may exhibit a tensile elongation in the ranges described herein.

The intermediate layer 226 may provide, in one or more embodiments, properties such as pressure distribution (where, for example, the intermediate layer is a foam layer, gel, lofted woven or nonwoven, etc.). In one or more embodiments, the intermediate layer 226 may also be a hydrophilic layer capable of absorbing and carrying liquids such as, e.g., water, normal saline, etc.

The outer covering 222 may be provided to protect the intermediate layer 226 of the coversheet 220. The outer covering 222 may, in one or more embodiments, also provide a coefficient of friction to the outer surface of the coversheet 220 that is within the selected ranges as discussed herein to limit abrasion on tissues and/or organs while retaining sufficient frictional properties to function as needed in restraining tissues and/or organs as discussed herein.

Although the outer covering 222, base layer 224 and intermediate layer 226 are described as having certain characteristics, it should be understood that any of the layers may have any of the described characteristics. For example, although the intermediate layer 226 is described as providing pressure distribution and/or being hydrophilic, one or both of the outer covering 222 and/or base layer 224 may possess these characteristics in place of or in addition to intermediate layer 226.

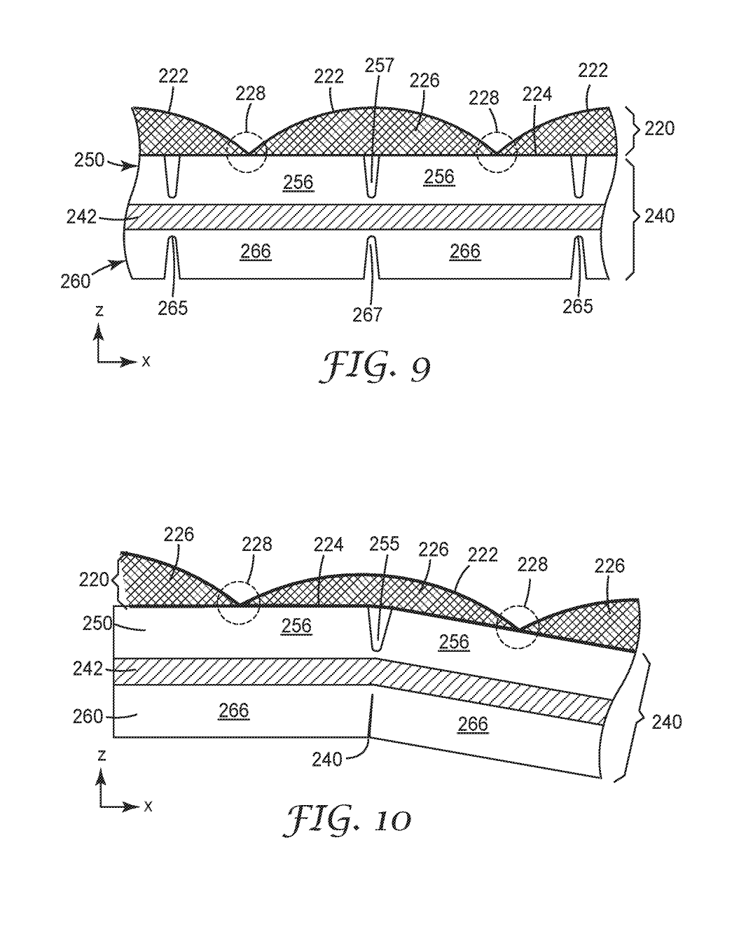

In one or more embodiments, the cover sheets may be attached over the entire exposed surfaces of the structured elements on a shapeable member of a shapeable article as described herein. In one or more alternative embodiments, however, the cover sheets may be attached only in selected areas on portions of the structured elements. One exemplary embodiment of such a construction is depicted in FIG. 9 where a coversheet 220 is attached to structured elements 256 in only selected areas 228 on portions of the surfaces of structured elements 256. Attachment of the coversheet 220 to the structured elements 256 may be accomplished using any suitable technique or combination of techniques as described herein

The attachment of coversheets onto a shapeable member in one or more embodiments of shapeable articles as described herein will preferably not unduly limit the ability of the shapeable member 240 to be plastically deformed as described herein. Interaction of the coversheet 220 with the underlying structured elements 256 of sheet 250 during deformation of the shapeable member 240 can be described with reference to FIGS. 9-11.

The combination of components in illustrative embodiment of coversheet 220 may provide, in one or more embodiments, advantages. For example, in one or more embodiments in which the base layer 224 provides elastic extensibility, manipulation of the shapeable member 240 to shapes such as depicted in FIGS. 10 and 11 may not be unduly limited.

Furthermore, providing a base layer 224 that spans openings between adjacent structured elements 256 may limit the ability of intermediate layer 226 to enter into the gap 257 between the structured elements 256 which could, in one or more embodiments, limit the range of movement of the structured elements towards each other. In particular, manipulation of the shapeable member 240 as depicted in FIGS. 10 and 11 may, in one or more embodiments, close the gaps 267 between adjacent structured elements 266 while opening the gap 257 between structured elements 256. If material were located in, e.g., gap 267, bending of the shapeable member 242 close gap 267 may be limited.

Another optional feature depicted in coversheet 220 is the configuration of outer covering 222. In particular, outer covering 222 is shown as taking a quilted configuration in which the outer covering 222 has a length between attachment points 228 that is greater than the straight line distance between attachment points 228 on adjacent structured elements 256 when the malleable core 242 is in a flat configuration. In such embodiments, the outer covering 222 may not, itself, need to exhibit extensibility as the shapeable member 240 takes on a curved configuration because the outer covering 222 may simply be pulled into closer proximity with the structured elements 256 as depicted in FIGS. 9, 10, and 11 without requiring extensibility in the outer covering 222.

Another optional feature that may improve the ability of the shapeable member 240 to be deformed is depicted in connection with FIG. 11 in which second sheet 260 has been manipulated such that a gap 267 no longer exists between adjacent structured elements 266. In embodiments where the structured elements 266 are, themselves, constructed of elastically deformable material, that material may be compressed and/or deformed as seen in areas 269 on structured elements 266 as the shapeable member 240 is bent around a surface 200.

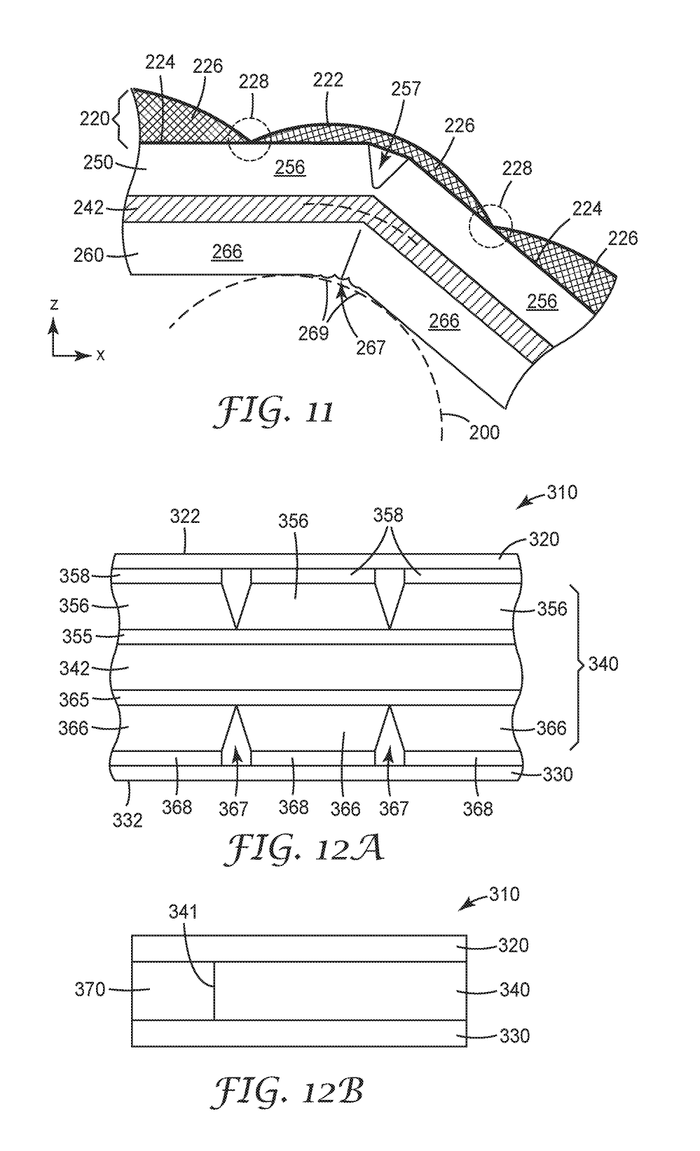

Another illustrative embodiment of a shapeable article 310 is depicted in a partial cross-sectional view in FIG. 12A. Shapeable article 310 includes a shapeable member 340 having a first cover sheet 320 attached to one major surface and a cover sheet 330 attached to an opposing major surface. The shapeable member 340 includes a malleable core 342 having a land portion 355 attached to a first major surface and a land portion 365 attached to a second major surface of the malleable core 342. Structured elements 356 are attached to land portion 355 on the first major surface of the malleable core 342, while structured elements 366 are attached to land portion 365 on the second major surface of the malleable core 342. The structured elements 356 are separated from each other by gaps 357 while the structured elements 366 are separated from each other by gaps 367 as described herein in connection with other illustrative embodiments of shapeable members.

One difference between the shapeable member 340 of shapeable article 310 is that each of the structured elements 356 includes a compressible layer 358 located between each of the structured elements 356 and the cover sheet 320. In one or more embodiments, the compressible layer 358 may be provided in the form of a foam, although other compressible materials may be used. Structured elements 366 on the opposite major surface of the shapeable member 340 may also include a similar compressible layer 366 located between each of the structured elements 366 and the cover sheet 330.

The compressible layers 358 and 368 on the structured elements 356 and 366 may, in one or more embodiments, assist in controlling the formation of pressure points on tissue and/or organs that come into contact with the shapeable article. In those embodiments in which the compressible layers 358 and 368 are provided in the form of a foam, the foam may be an open or closed cell foam. Any compressible layer used on individual structured elements as described herein may, in one or more embodiments, exhibit properties similar to those described above for compressible layers used in a coversheet on a shapeable article as described herein.

Attachment of the cover sheets 320 and 330 to the compressible layers 358 and 368 may be achieved by any suitable technique or combination of techniques as described herein for the coversheets described above.

Another optional feature that may be provided in one or more embodiments of shapeable articles as described herein is depicted and will be described in connection with FIG. 12B, which depicts an enlarged cross-sectional view of a portion of one illustrative embodiment of a shapeable article as described herein. As seen in FIG. 12B, the shapeable article 310 includes a shapeable member 340, a first coversheet 320, and a second coversheet 330. Both of the cover sheets 320 and 330 are attached to the shapeable member 340 as described elsewhere herein.

The additional optional feature depicted in FIG. 12B is an edge protector 370. In the depicted illustrative embodiment, the edge protector 370 is positioned proximate a perimeter 341 of the shapeable member 340. In one or more embodiments, at least a portion of the edge protector 370 is located between the first cover sheet 320 and the second cover sheet 330. Although the depicted illustrative embodiment of edge protector 370 depicts the edge protector 370 as having an exposed edge outside of the perimeter 341 of the shapeable member 340, in one or more alternative embodiments, one or both of the cover sheets 320 and/or 330 may extend past the edge protector 370 such that the edge protector 370 can be encased within one or both of the cover sheets 320 and/or 330 about the perimeter 341 of the shapeable member 340.

Further, it should be understood that edge protectors used in one or more embodiments of shapeable articles as described herein may take a variety of shapes. For example, although the edge protector 370 in the depicted illustrative embodiment has a generally rectangular shape, other embodiments of edge protectors may take any suitable shape. For example, in one or more embodiments, an edge protector used in one or more embodiments of a shapeable article as described herein may include a slot or channel capable of receiving an edge at the perimeter of a shapeable member as described herein.

In one or more embodiments, the edge protector 370 extends about at least a portion of the perimeter 341 of the shapeable member 340. In one or more embodiments, the edge protector 370 may extend about only a portion of the perimeter 341 of the shapeable member 340 which may be used in one or more embodiments of a shapeable article as described herein. For example, in one or more embodiments, the edge protector 370 may be located along only one side of a generally rectangular shapeable article 310. In one or more alternative embodiments, the edge protector 370 may extend around the entire perimeter 341 of a shapeable member 340 used in one or more embodiments of a shapeable article as described herein.

Edge protectors used in connection with one or more embodiments of a shapeable article as described herein may provide additional protection for tissue that contacts an edge of a shapeable article as described herein. In one or more embodiments, the edge protectors used in connection with one or more embodiments of a shapeable article as described herein may be formed of one or more compressible materials such as those described herein with respect to cover sheets used in shapeable articles as described herein. In one or more embodiments, the edge protectors used in connection with one or more embodiments of a shapeable article as described herein may be formed of one or more compressible materials such as, e.g., those described herein with respect to cover sheets used in shapeable articles as described herein.

Edge protectors used in connection with one or more embodiments of shapeable articles as described herein may be attached to one or both of the cover sheets and/or the shapeable member used in the shapeable article by any suitable technique or combination of techniques. For example, in one or more embodiments, one or more adhesives may be used to attach the edge protectors to cover sheets and/or a shapeable member of a shapeable article. Potentially useful adhesives may, in one or more embodiments, include a pressure sensitive adhesive such as, e.g., an acrylate, polyurethane, polyolefin, styrene copolymer or a combination thereof; a hot melt adhesive such as a polyolefin or modified polyolefin (ethylene vinylacetate, ethylene acrylates such as ethylene methylacrylate, acrylates such as KURARITY (from Kuraray) and the like), or a curable adhesive such a 2 part silicone, epoxy, or polyurethane. Alternatively, or in addition to adhesives, the edge protectors used in one or more embodiments of shapeable articles as described herein may be attached to one or both of the cover sheets and/or the shapeable member using one or more other techniques such as, e.g., welding (one or more of thermal, chemical, and mechanical welding), sewing, mechanical fasteners (e.g., hook and loop fasteners, stem fasteners (e.g., 3M DUAL LOCK reclosable stem fasteners), etc.), riveting, stitching, crimping, etc.

The structured elements and land portions on sheets used in shapeable members of shapeable articles as described herein may, in one or more embodiments, take a variety of different shapes and/or arrangements. For example, as seen in FIG. 4, all of the structured elements 256 have the same shape as well as the same size. In one or more alternative embodiments, however, the structured elements on a first major surface of a malleable core of a shapeable member of a shapeable article as described herein may have different shapes and/or different sizes. Furthermore, in one or more embodiments, the structured elements on a second major surface of the malleable core may also be provided with the same shape as well as the same size, although in one or more alternative embodiments the structured elements on a second major surface of a malleable core may have different shapes and/or different sizes from the structured elements on the first major surface.

As also seen in the illustrative embodiment depicted in FIG. 4, the structured elements on one major surface of a malleable core of a shapeable member of a shapeable article as described herein are arranged in a uniformly spaced array, e.g., a rectilinear array or other arrangement. In one or more alternative embodiments, however, the structured elements on malleable cores of shapeable members used in shapeable articles as described herein may not be arranged in a uniformly spaced array.

Further, although the structured elements on one major surface of a malleable core of a shapeable member of a shapeable article as described herein may have the same size and shape, the structured elements on sheets used in shapeable members of shapeable articles as described herein may take a wide variety of different shapes and/or sizes. Some illustrative examples of sheets having structured elements with different sizes and/or shapes are depicted in FIGS. 13-16.

As seen in FIG. 13, the depicted surface of shapeable member 340 includes a land portion 355 separating a variety of differently shaped structured elements 356a, 356b, and 356c. Another feature depicted in connection with the illustrative embodiment of shapeable member 340 is seen in the distribution of areas 328 in which a cover sheet could be attached to the structured elements. In particular, only selected structured elements 356a may, in the depicted embodiment, be attached to a cover sheet. Such an arrangement may, in one or more embodiments, improve flexibility of the shapeable article formed using shapeable member 340.

Another illustrative embodiment of a shapeable member 340 that may be used in one or more embodiments of a shapeable article as described herein is depicted in FIG. 14. The shapeable member 340 in FIG. 14 includes structured elements 356 in the form of hexagonal shapes separated by land portions 355. Further, only selected structured elements 356 may, in one or more embodiments, be attached to a cover sheet position over the shapeable member 340 in areas 328.

Still another illustrative embodiment of a shapeable member 340 is depicted in FIG. 15 and includes structured elements 356 separated by a land portion 355. Again, only selected structured elements 356 of the shapeable member 340 may be attached to a cover sheet position over the shapeable member 340 in areas 328.

While structured elements depicted in, e.g., FIGS. 4, 13, and 14 are in the form of polygonal shapes, the structured elements 356 depicted in FIG. 15 are in the form of circles. It should, however, be understood that structured elements used in shapeable members as described herein may take any suitable shape or shapes, e.g., polygonal, circular, oval, elliptical, etc., as well as composite shapes, e.g., shapes that combine one or more shapes selected from polygonal, circular, oval, elliptical, etc. shapes. Furthermore, structured elements on a major surface of a malleable core of any given shapeable member may include a mixture of both polygonal and non-polygonal shape structured elements.

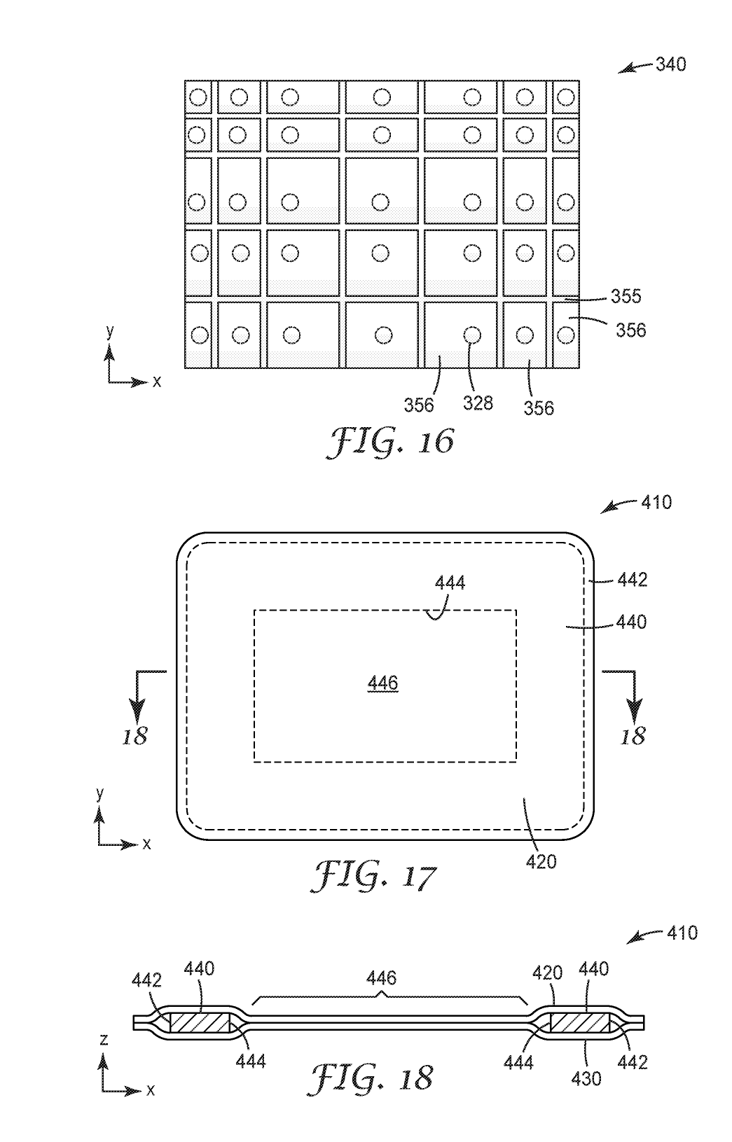

Yet another variation in structured elements on a sheet that may be used in one or more embodiments of a shapeable member used in a shapeable article as described herein is depicted in FIG. 16. Among the variations depicted in FIG. 16 are differences in the sizes of the structured elements 356. In one or more embodiments, providing structured elements 356 having smaller dimensions may improve the flexibility of the shapeable member 340 in areas occupied by the smaller structured elements. Unlike the shapeable member 340 depicted in FIGS. 13 to 15, all of the structured elements 356 on shapeable member 340 include an attachment area 328 where a cover sheet may be attached to the shapeable member 340.

Although in one or more embodiments, the shapeable members in shapeable articles as described herein may be completely continuous within their outer perimeters, one or more alternative embodiments may include shapeable members having one or more window openings formed within an outer perimeter. One illustrative example of a shapeable article 410 including a shapeable member 440 is depicted in FIGS. 17 and 18, where FIG. 18 is a cross-sectional view of shapeable article 410 taken along line 18-18 in FIG. 17. The shapeable article 410 includes coversheets 420 and 430 located on opposite sides of the shapeable member 440. Coversheets 420 and 430 may be similar to any of the coversheets described in connection with other illustrative embodiments of shapeable articles as described herein. Similarly, shapeable member 440 may be constructed of components as discussed herein with respect to other shapeable members in other illustrative embodiments of shapeable articles as described herein.

The shapeable member 440 includes an outer perimeter 442 inset from an outer perimeter 416 of the shapeable article 410 such that the coversheets 420 and 430 form a border around the outer perimeter 442 of the shapeable member 440. In addition to outer perimeter 442 shapeable member 440 also includes an inner perimeter 444 defining a window opening 446 within the boundary of the outer perimeter 442 of the shapeable member 440.

Although shapeable member 440 includes only one window opening 446, one or more alternative embodiments of shapeable articles as described herein may include shapeable members having two or more separate window openings located within the boundary of the outer perimeter 442 of the shapeable member 440. Further, although the inner perimeter 444 and corresponding window opening 446 have a shape similar to the shape of the outer perimeter 442 of the shapeable member 440, window openings with shapes other than those of the outer perimeter 442 of shapeable member 440 may also be used in shapeable articles as described herein. In addition, the shape of the inner perimeter 444 and its corresponding window opening 446 are not required to be rectangular as depicted in FIG. 17, i.e., the inner perimeter 444 and its corresponding window opening 446 may take any selected shape.

Another optional feature depicted in connection with shapeable article 410 is that one or both of the coversheets 420 and 430 extend over the window opening 446 formed in the shapeable member 440. As a result, although the shapeable member 440 includes a window opening 446, the shapeable article 410 in which shapeable member 440 is located does not include a window opening because that opening is closed by one or more of the coversheets 420 and/or 430.

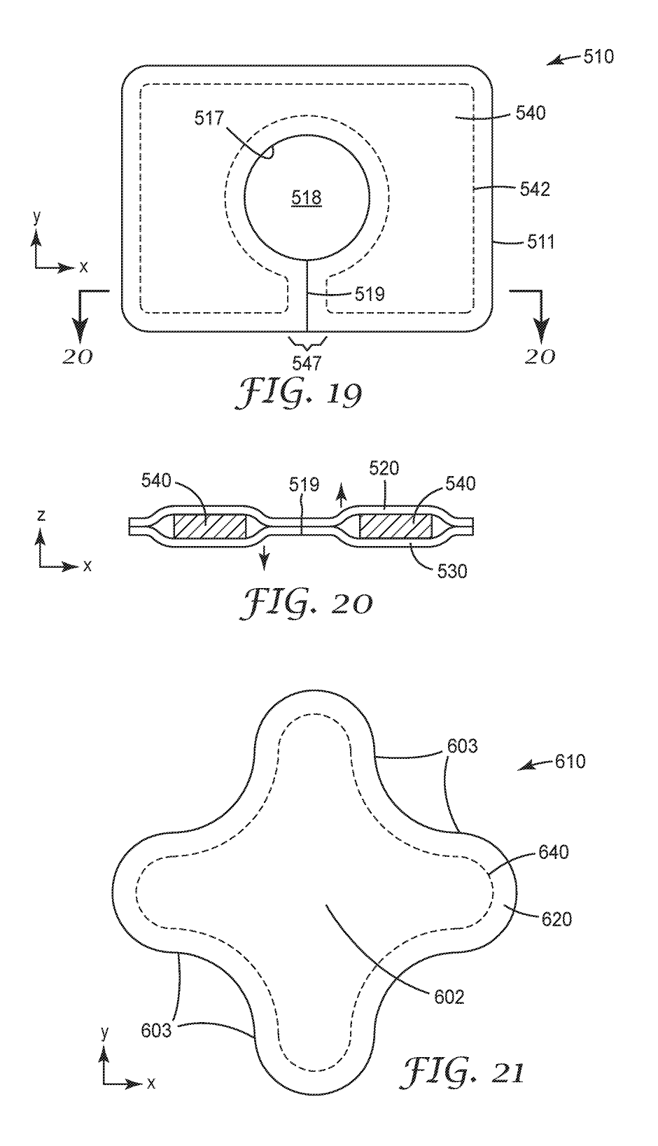

Another illustrative embodiment of a shapeable article 510 including a shapeable member 540 is depicted in FIGS. 19 and 20, where FIG. 20 is a cross-sectional view of the shapeable article 510 taken along line 20-20 in FIG. 19. The shapeable article 510 includes a shapeable member 540 located between coversheets 520 and 530. In this illustrative embodiment, the coversheets 520 and 530 include an inner perimeter 517 defining a window opening 518 in the coversheets 520 and 530 in addition, the shapeable member 540 includes a perimeter 542 that follows the shape of the inner perimeter 517 such that the shapeable article 510 includes a window opening 518 formed through both the coversheets 520 and 530 as well as the shapeable member 540.

Another optional feature depicted in connection with the illustrative embodiment of shapeable article 510 is an access slit 519 formed through the coversheets 520 and 530 to allow access from an outer perimeter 511 of the shapeable article 510 to the window opening 518 defined by inner perimeter 517. In the illustrative embodiment depicted in FIGS. 19 and 20, the shapeable member 540 includes an outer perimeter 542 that follows outer perimeter 511 of the shapeable article 510, but extends inwardly on both sides of the access slit 519 where the outer perimeter 542 of the shapeable member 540 follows inner perimeter 517 defining window opening 518. As a result, the shapeable member 540 forms a gap 547 leading to window opening 518.

The access slit 519 formed through the coversheets 520 and 530 may be in the form of a cut line formed through the coversheets 520 and 530, with the coversheets 520 and 530 separated along the access slit 519. In one or more alternative embodiments, however, access slit 519 may be in the form of a row of perforations, a line of weakness, etc. along which coversheets 520 and 530 preferentially separate. In one or more embodiments, the shapeable article 510 can be manipulated on both sides of the access slit 519 to provide an opening through which tissues, organs, etc. can be inserted into the window opening 518. In one or more embodiments, manipulation of the shapeable article 510 in opposite directions on opposite sides of the access slit 519 as depicted by the arrows seen in FIG. 20 may be used to provide such an opening for access to the window opening 518.

Although shapeable articles as described herein may often take the shape of a rectangle, shapeable articles may, in one or more alternative embodiments take a variety of different shapes, one of which is depicted in FIG. 21. The illustrative embodiment of shapeable article 610 depicted in FIG. 21 is one example of a shapeable article having a nonrectangular shape. In particular, shapeable article 610 includes a central portion 602 with fingers 603 extending outward from the central portion 602. In one or more embodiments, the shapeable member 640 provided in the shapeable article 610 extends into each of the fingers 603 such that each of the fingers 603 can be manipulated into and maintain a non-planar shape along with central portion 602.

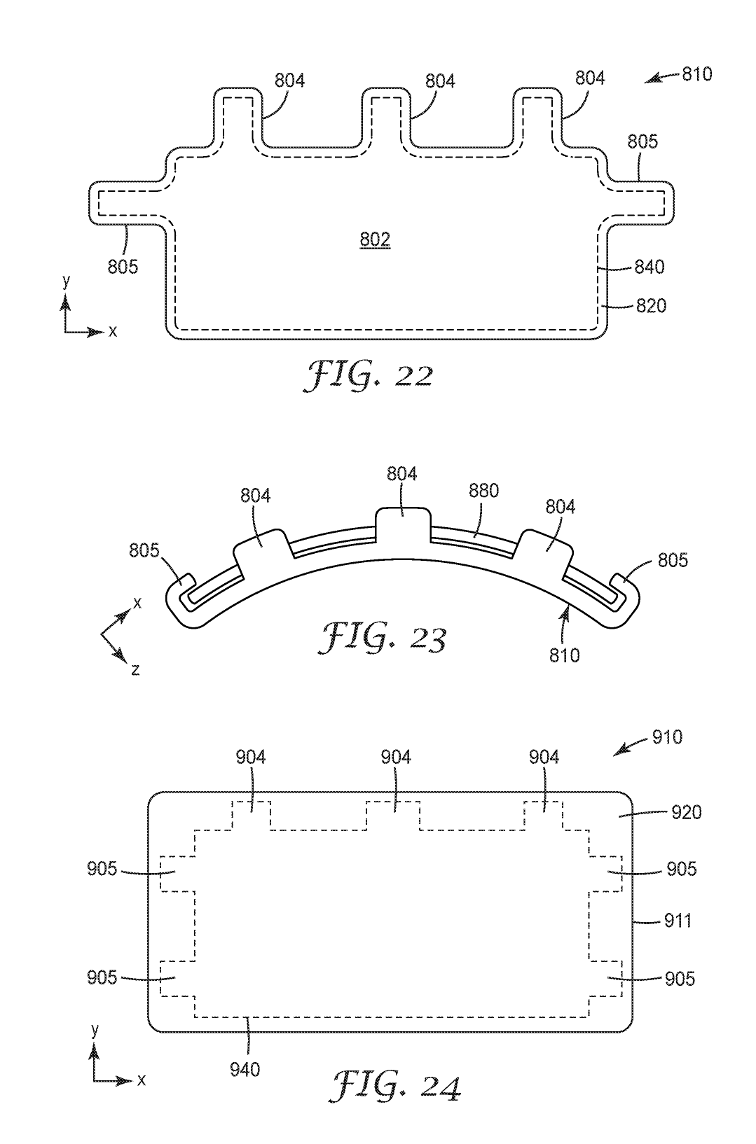

Another illustrative embodiment of a shapeable article as described herein is depicted in FIG. 22 where shapeable article 810 includes a shapeable member 840 located within a pair of coversheets, with only one coversheet 820 seen in the view of FIG. 22. Shapeable article 810 includes a central portion 802 in the general shape of a rectangle, while tabs 804 extend away from the generally rectangular central portion 802 along one side. Further, tabs 805 extend away from the generally rectangular central portion along opposite sides of the rectangular central portion 802. As seen in the shapeable article 810, tabs 804 and 805 which extend away from the generally rectangular central portion 802 do not occupy all of the side of the rectangular shape central portion from which they extend. In other words, the tabs 804 and 805 occupy less than all of the side from which they extend.

In the illustrative embodiment depicted in FIG. 22, the shapeable member 840 extends into the area defined by each of the tabs 804 and 805. In one or more alternative embodiments, however, the shapeable member may or may not extend into those tabs. Further, in one or more alternative embodiments the shapeable member may or may not occupy substantially all of the central portion 802 of the shapeable article 810 as depicted in FIG. 22. It should be understood that the arrangement of tabs 804 and 805 and shapeable article 810 provide only one example of an arrangement of tabs that may be provided on shapeable articles as described herein.

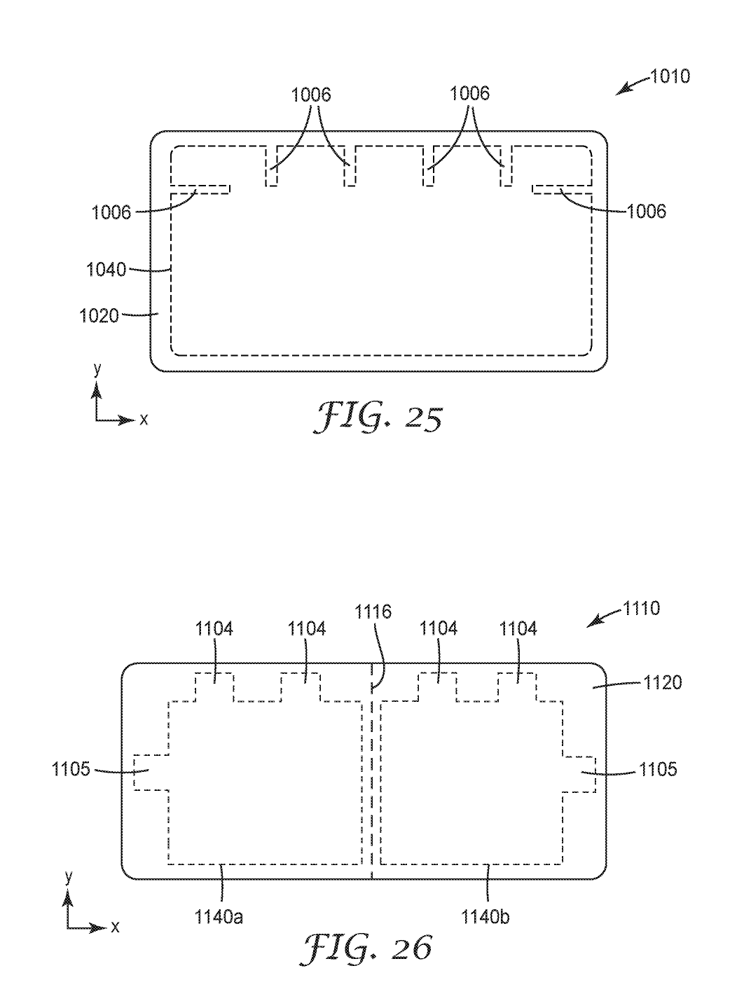

Shapeable articles such as, e.g., illustrative embodiment of shapeable article 810, that include tabs may be well-suited for use in attaching the shapeable article 810 to other structures to assist in restraint of tissues and/or organs during surgical procedures. For example, the shapeable article 810 is depicted in FIG. 23 as being deployed on a curved retractor plate 880 which may be used in, for example, a table-mounted retraction system (e.g., a BOOKWALTER retraction system). As depicted, the tabs 804 may be bent or manipulated over a top/bottom edge of the retractor plate 880 while tabs 805 may be bent or folded over a side edge of the retractor plate 880 such that shapeable article 810 may be retained in a selected position on the retractor plate 880.

Still another illustrative embodiment of a shapeable article as described herein is depicted in FIG. 24 where shapeable article 910 is depicted. Shapeable article 910 includes coversheets attached to and containing a shapeable member 940 as described herein in connection with other illustrative embodiments of shapeable articles (only one coversheet 920 is visible in FIG. 24). The coversheets of shapeable article 910 define an outer perimeter 911 for the shapeable article that is, in the depicted illustrative embodiment, generally in the shape of a rectangle.

The shapeable member 940 contained within the coversheets of the shapeable article 910, however, has a different shape from the shape defined by the outer perimeter 911. In particular, shapeable member 940 includes a central section 942 with tabs 904 and 905 extending outwardly from multiple sides of the central portion 942 of the shapeable member 940. In other words, although the shapeable article 910 may be described as including tabs, those tabs do not protrude from an outer perimeter of the shapeable article 910 as depicted above in connection with FIG. 22. Regardless, the tabs 904 and 905 of shapeable member 940 in shapeable article 910 may be used to assist in securing the shapeable article 910 on other structures in a manner similar to that depicted in FIG. 23 in connection with shapeable article 810.

Shapeable article 910 provides one example in which coversheets containing a shapeable member of a shapeable article define an article perimeter 911, while the shapeable member 940 located between the coversheets has a shapeable member perimeter, where the article perimeter and the shapeable member perimeter are different from each other. This is in contrast with, e.g., shapeable articles depicted in FIGS. 1, 17, 19, 21, and 22 in which the article perimeters and the shapeable member perimeters have the same shape (although the member perimeter is typically inset from the article perimeter to allow for a border of coversheet material to encase the shapeable member located between coversheets).

Another illustrative embodiment of a shapeable article as described herein is depicted in FIG. 25. Shapeable article 1010 includes a shapeable member 1040 located between coversheets as discussed herein with respect to other illustrative embodiments of shapeable articles (with only one coversheet 1020 visible in the view of FIG. 25). The shapeable member 1040 includes one or more slits which, in the depicted embodiment, includes multiple slits 1006 that extend from an edge of the shapeable member 1040 inward toward a center of the shapeable member 1040. The slits may be provided to increase flexibility of the shapeable member around its perimeter.

Another illustrative embodiment of a shapeable article as described herein is depicted in FIG. 26. Shapeable article 1110 includes two shapeable members 1140a and 1140b located between coversheets (with only one coversheet 1120 visible in the view of FIG. 25). Each of the two shapeable members, which will be commonly referred to as shapeable members 1140, includes, in the depicted illustrative embodiment, tabs 1104 and 1105 extending outwardly from a central portion of each of the shapeable members 1140.

An additional feature depicted in connection with illustrative embodiment of shapeable member 1110 is a separation line 1116 that extends across the shapeable member 1110 between the shapeable members 1140. Separation line may be used to reduce the size of the shapeable article 1110 by allowing for separation of the coversheets along separation line 1116 while maintaining containment of the shapeable members 1140 between the coversheets after separation of the coversheets along separation line 1116. Separation line 1116 may be provided in a variety of forms, e.g., a plurality of perforations extending along line 1116, one or more lines of weakness in one or both of the coversheets containing separation line 1116, etc. In still other embodiments, separation line 1116 may merely be provided with printed or otherwise visible indicia indicating where a user may cut the coversheets without cutting or exposing the shapeable members 1140 located within the coversheets.

Although only one separation line 1116 and two shapeable members 1140 are depicted in the illustrative embodiment of shapeable article 1110, one or more alternative embodiments of shapeable articles as described herein may include two or more separation lines and a corresponding number of shapeable members (for example, a shapeable article including two separation lines may, in one or more embodiments, include three separate shapeable members located within the coversheets).

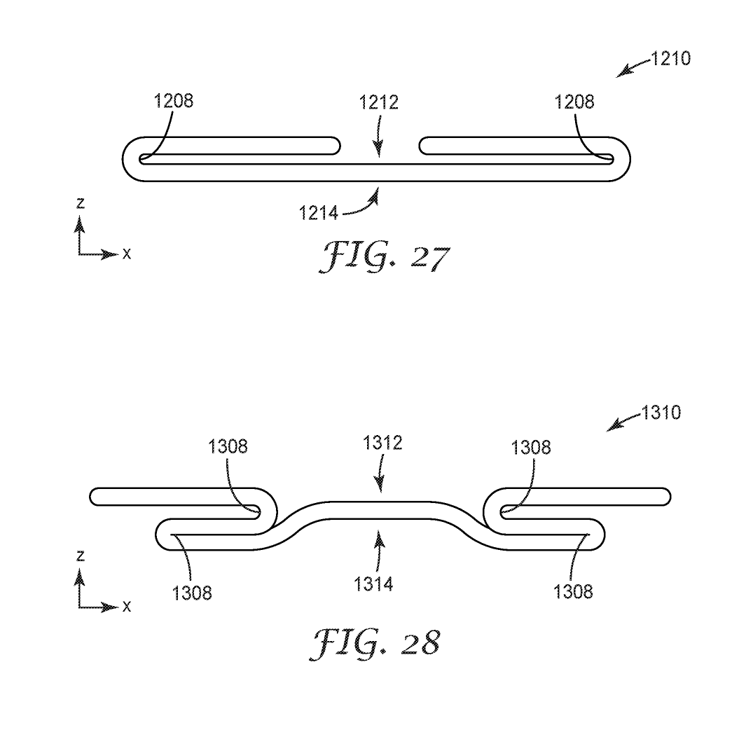

While illustrative embodiment of shapeable article 1110 depicted in FIG. 26 provides one example of a shapeable article that can be reduced in size using, e.g., separation line 1116, the illustrative embodiments of shapeable articles depicted in FIGS. 27 and 28 provide one example of features that may be provided to increase the size of a shapeable article.

In particular, shapeable article 1210 as depicted in FIG. 27 includes first and second major surfaces 1212 and 1214. The shapeable article 1210 also includes two fold lines 1208, with one or both of the fold lines 1208 providing for expansion of the first and second major surfaces 1212 and 1214 by unfolding of the shapeable article along one or both of the fold lines 1208. If, for example, fold-line 1208 on the right side of the shapeable article 1210 were unfolded, the size of the first and second major surfaces 1212 and 1214 would be increased because more of the shapeable article 1210 would be facing both upward and downward in the view depicted in FIG. 27.

Another illustrative embodiment of shapeable article 1310 as depicted in FIG. 28 provides a different set of fold lines that may be used to increase the size of the first and second major surfaces 1312 and 1314 of the shapeable article 1310. In particular, shapeable article 1310 includes multiple fold lines 1308. In one or more embodiments, one or more of these fold lines may be unfolded to increase the size of the first and second major surfaces 1312 and 1314. Unfolding of this embodiment may potentially be achieved by, e.g., placing the shapeable article in tension along the X direction.

The shapeable articles as described herein may be used in a variety of surgical procedures to restrain tissues and/or organs from entering or leaving a selected area. In one or more embodiments, the shapeable articles as described herein may be combined by folding and/or interlocking complementary features found on two or more of the shapeable articles as described herein.

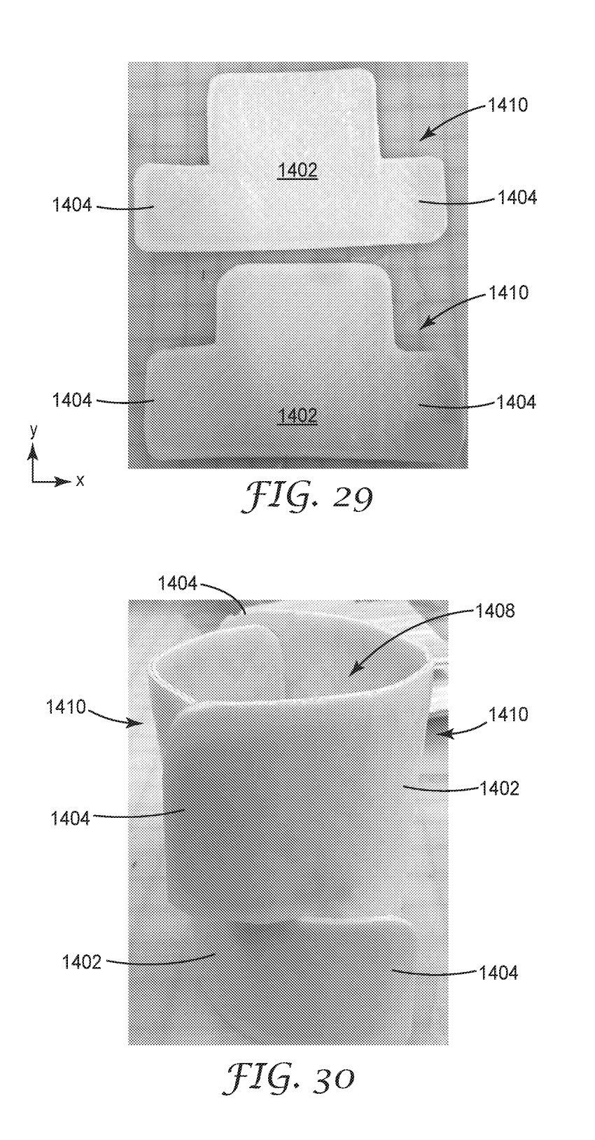

FIG. 29 depicts one illustrative embodiment of shapeable articles 1410 that may be used together in a manner that helps to define an area from which tissue and/or organs may be restricted from entering or leaving. Each of the shapeable articles 1410 includes a central portion 1402 and a pair of tabs 1404 extending away from the central portion on opposite sides of central portion 1402 of the shapeable article 1410.

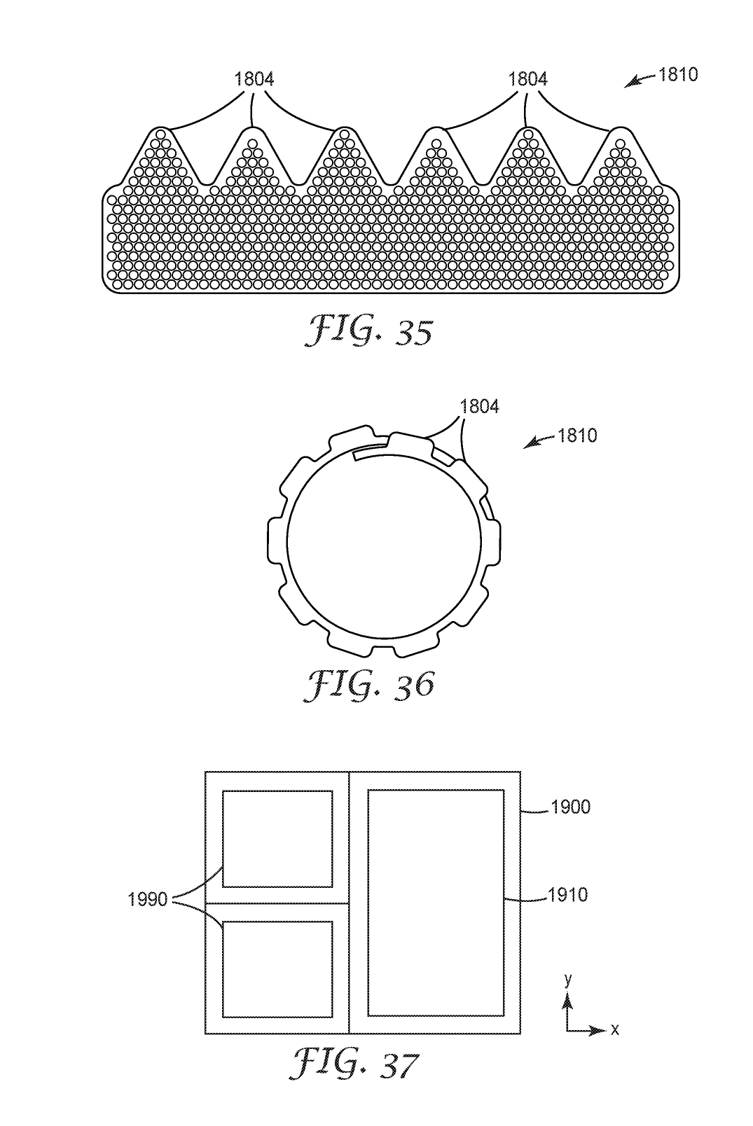

As seen in FIG. 30, the two shapeable articles 1410 depicted in FIG. 36 have been manipulated into arcuate shapes and arranged such that tabs 1404 on opposing shapeable articles 1410 overlap with the central portions 1402 of the opposing shapeable article 1410 to form a loosely interlocking relationship defining an area or passage 1408 between the central portions 1402 of the pair of shapeable articles 1410. Tissues and/or organs may be restricted from entering or leaving area or passage 1408 when the pair of shapeable articles 1410 depicted in FIG. 30 are used in a surgical procedure.

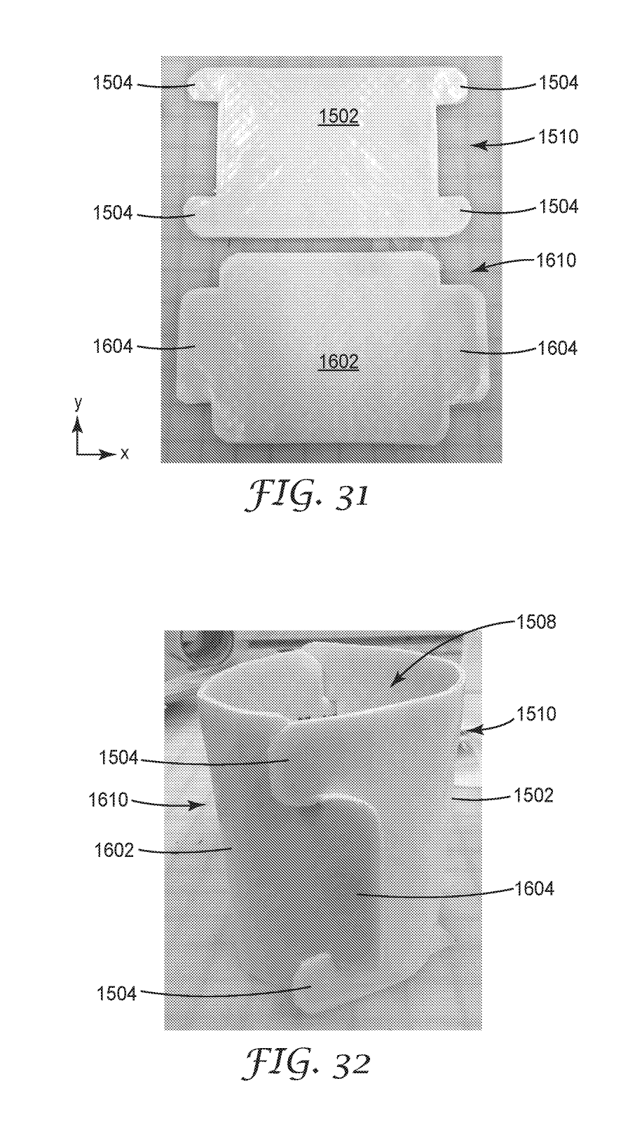

FIG. 31 depicts another illustrative embodiment of two different shapeable articles 1510 and 1610 that may be used together in a manner to help define an area or passage. Shapeable article 1510 includes a central portion 1502 with a pair of tabs 1504 extending from two sides of the generally rectangular central portion. Shapeable article 1610 includes a generally rectangular central portion 1602 with a single tab extending from each of the two opposing edges of the generally rectangular central portion 1602.

In one or more embodiments, the tabs 1504 extending from one edge of the shapeable article 1510 may be separated from each other by a distance that corresponds generally to the length of tab 1604 extending from one edge of shapeable article 1610. As a result, at least some mechanical interference may be achieved when tab 1604 on shapeable article 1610 is positioned between tabs 1504 on shapeable article 1510. Mechanical interference between tab 1604 and pair of tabs 1504 may be enhanced where, for example, the length of tab 1604 on shapeable article 1610 is equal to or greater than the distance between the pair of tabs 1504 on shapeable article 1510.

As seen in FIG. 32, the two shapeable articles 1510 and 1610 depicted in FIG. 31 have been manipulated into arcuate shapes and arranged such that each of tabs 1604 on shapeable article 1610 is positioned between a pair of tabs 1504 on shapeable article 1510. The pair of shapeable articles 1510 and 1610, when so manipulated and arranged, may provide an area or passage 1508 between their respective central portions 1502 and 1602. Tissues and/or organs may be restricted from entering or leaving the area or passage when the pair of shapeable articles 1510 and 1610 depicted in FIG. 32 are used in a surgical procedure.

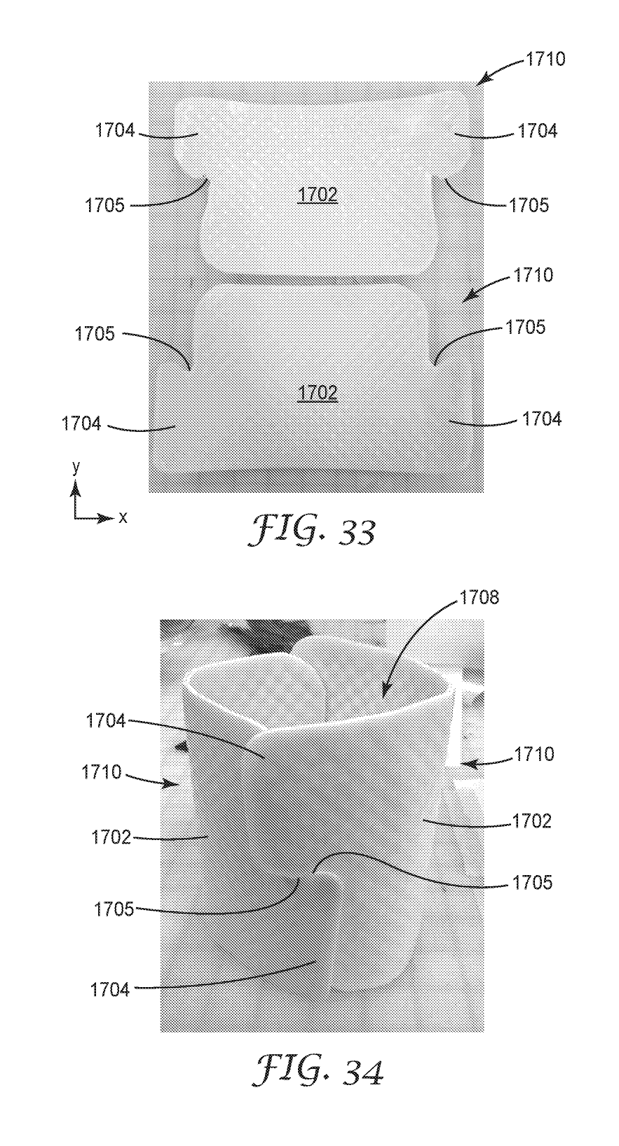

FIG. 33 depicts another illustrative embodiment of a shapeable article 1710 that may be used in groups of two or more to define an area or passage from which, e.g., tissues and/or organs may be restricted from entering or leaving during a surgical procedure. In particular, the shapeable articles 1710 include a central portion 1702 with tabs 1704 extending from opposing edges of the shapeable article 1710. An additional feature depicted in connection with shapeable article 1710 are notches 1705 formed at a junction of the tabs 1704 on the intermediate end of the tabs 1704 (i.e., the end of the tabs 1704 located between opposing edges of the central portion 1702 rather than at a corner of the shapeable article 1710).

As seen in FIG. 34, the two shapeable articles 1710 depicted in FIG. 33 have been manipulated into arcuate shapes and arranged such that tabs 1704 on opposing shapeable articles 1710 overlap with the central portions 1702 of the opposing shapeable article 1710. The interlocking relationship between the pairs of tabs 1704 on the opposing shapeable articles 1710 is enhanced by the complementary notches 1705 located on the intermediate ends of the tabs 1704 and may, in one or more embodiments, provide more resistance to disengagement of the shapeable article 1710 from each other during use. The opposing shapeable article 1710, when arranged and interconnected as seen in FIG. 34, define an area or passage 1708 between the shapeable article 1710 from which tissues and/or organs may be restricted from entering or leaving when the pair of shapeable articles 1710 are used in a surgical procedure.

FIG. 35 depicts another illustrative embodiment of a shapeable article 1810 that may be used to define an area or passage from which, e.g., tissues and/or organs may be restricted from entering or leaving during a surgical procedure. In particular, the shapeable articles 1810 includes a central portion 1802 with tabs 1804 extending from an edge of the shapeable article 1810.

As seen in FIG. 36, the shapeable article 1810 depicted in a flat configuration in FIG. 35 has been manipulated into an arcuate shape and arranged such that tabs 1804 on the shapeable article 1810 overlap with the central portion 1802 to assist in holding the arcuate shape of the 1810 as depicted in FIG. 36. Alternatively, the tabs 1804 may be bent over the edge of an incision and/or other retraction devices (e.g., retraction plates in a table-mounted retraction system, etc.).

Although some illustrative embodiments of shapeable articles as described herein rely on folding or bending of a shapeable member located in the shapeable article as discussed in connection with, e.g., FIG. 23 or mechanical interference between two shapeable articles to retain a shape or position as discussed in connection with, e.g., FIGS. 29-34, or mechanical interference caused by folding of one shapeable article as discussed in connection with FIGS. 35-36, one or more embodiments of shapeable articles as described herein may include other components that may be used to assist in attaching the shapeable articles to other structures and/or surfaces (e.g., retractor plates, surgical drapes, incise drapes, wound edge protectors, patient's skin, etc.) and/or to each other/themselves. In particular, one or more embodiments of shapeable articles as described herein may include pressure sensitive adhesives located on at least a portion of an exterior surface of the shapeable article, cohesive materials on at least a portion of an exterior surface of the shapeable article, mechanical fasteners (e.g., hook and loop fasteners, stem fasteners (e.g., 3M DUAL LOCK reclosable stem fasteners), magnets, etc.

One or more of the shapeable articles described herein may be provided in a kit along with one or more other items. FIG. 37 is a schematic diagram of one illustrative kit that includes at least one shapeable article 1910 as described herein along with other articles such as e.g. lap sponges 1990. All of the components of the kit may be contained within a package 1900 which may, in one or more embodiments, be a sealed package, with the articles located therein being sterilized for use in a surgical procedure. In still other embodiments, the package 1900 may be a frangible sealed package containing sterilized items such as one or more shapeable articles 1910 and one or more lap sponges 1990.

### ILLUSTRATIVE EMBODIMENTS

### Embodiment 1

A manually shapeable article comprising: a shapeable member comprising: a malleable core comprising a first major surface and a second major surface, wherein the first major surface and the second major surface are on opposite sides of the malleable core; a plurality of first structured elements attached to the first major surface of the malleable core, wherein each first structured element of the plurality of first structured elements comprises a protrusion extending away from the malleable core, wherein the plurality of first structured elements are spaced apart from each other by an inter-element distance when the malleable core is in a flat configuration, and wherein the inter-element distance between the plurality of first structured elements changes when the malleable core is deformed into a non-flat configuration; optionally, a plurality of second structured elements attached to the second major surface of the malleable core, wherein each second structured element of the plurality of second structured elements comprises a protrusion extending away from the malleable core, wherein the plurality of second structured elements are spaced apart from each other by an inter-element distance when the malleable core is in a flat configuration, and wherein the inter-element distance between the plurality of second structured elements changes when the malleable core is deformed into a non-flat configuration; a first coversheet attached to the shapeable member, wherein the first coversheet covers the plurality of first structured elements of the shapeable member; and a second coversheet attached to the shapeable member, wherein the second coversheet covers the plurality of second structured elements of the shapeable member, such that the malleable core is located between first and second coversheets.

### Embodiment 2

An article according to embodiment 1, wherein the plurality of first structured elements are attached to each other by a land portion extending between the plurality of first structured elements.

### Embodiment 3

An article according to embodiment 2, wherein the land portion extending between the plurality of first structured elements is attached to the first major surface of the malleable core.

### Embodiment 4

An article according to any one of embodiments 2 to 3, wherein each first structured element of the plurality of first structured elements comprises a maximum element thickness measured transverse to the first major surface of the malleable core when the malleable core is in a flat configuration, wherein the element thickness is greater than a land thickness of the land portion extending between the plurality of first structured elements.

### Embodiment 5

An article according to embodiment 4, wherein the land thickness of the land portion extending between all of the first structured elements of the plurality of first structured elements is uniform.

### Embodiment 6

An article according to embodiment 4, wherein the land thickness of the land portion extending between two or more of the first structured elements of the plurality of first structured elements is different.

### Embodiment 7

An article according to any one of embodiments 1 to 6, wherein the structured elements of two or more neighboring structured element pairs of the plurality of first structured elements are separated from each other by an inter-element distance when the malleable core is in a flat configuration, wherein the inter-element distance is uniform when moving away from the malleable core.

### Embodiment 8

An article according to any one of embodiments 1 to 6, wherein the structured elements of two or more neighboring structured element pairs of the plurality of first structured elements are separated from each other by an inter-element distance when the malleable core is in a flat configuration, wherein the inter-element distance increases when moving away from the malleable core.

### Embodiment 9

An article according to any one of embodiments 1 to 8, wherein the plurality of second structured elements are attached to each other by a land portion extending between the plurality of second structured elements.

### Embodiment 10

An article according to embodiment 9, wherein the land portion extending between the plurality of second structured elements is attached to the second major surface of the malleable core.

### Embodiment 11

An article according to any one of embodiments 9 to 10, wherein each second structured element of the plurality of second structured elements comprises a maximum element thickness measured transverse to the second major surface of the malleable core when the malleable core is in a flat configuration, wherein the element thickness is greater than a land thickness of the land portion extending between the plurality of second structured elements.

### Embodiment 12

An article according to embodiment 11, wherein the land thickness of the land portion extending between all of the second structured elements of the plurality of second structured elements is uniform.

### Embodiment 13

An article according to embodiment 11, wherein the land thickness of the land portion extending between two or more of the second structured elements of the plurality of second structured elements is different.

### Embodiment 14

An article according to any one of embodiments 1 to 13, wherein the structured elements of two or more neighboring structured element pairs of the plurality of second structured elements are separated from each other by an inter-element distance when the malleable core is in a flat configuration, wherein the inter-element distance is uniform when moving away from the malleable core.

### Embodiment 15

An article according to any one of embodiments 1 to 13, wherein the structured elements of two or more neighboring structured element pairs of the plurality of second structured elements are separated from each other by an inter-element distance when the malleable core is in a flat configuration, wherein the inter-element distance increases when moving away from the malleable core.

### Embodiment 16

An article according to any one of embodiments 1 to 15, wherein the inter-element distance between the first structured elements comprises a maximum of 2 centimeters or less.

### Embodiment 17

An article according to any one of embodiments 1 to 16, wherein the inter-element distance between the first structured elements comprises a maximum of 1 centimeter or less.

### Embodiment 18

An article according to any one of embodiments 1 to 17, wherein the plurality of first structured elements are aligned with the plurality of second structured elements.

### Embodiment 19

An article according to any one of embodiments 9 to 18, wherein the land portion extending between the plurality of first structured elements is aligned with the land portion extending between the plurality of second structured elements.

### Embodiment 20

An article according to any one of embodiments 43 to 64, wherein all of the first structured elements of the plurality of first structured elements have the same shape.

### Embodiment 21

An article according to any one of embodiments 1 to 20, wherein all of the second structured elements of the plurality of second structured elements have the same shape.

### Embodiment 22

An article according to any one of embodiments 1 to 21, wherein all of the first structured elements of the plurality of first structured elements have the same size.

### Embodiment 23

An article according to any one of embodiments 1 to 21, wherein two or more of the first structured elements of the plurality of first structured elements have different shapes.

### Embodiment 24

An article according to any one of embodiments 1 to 23, wherein all of the second structured elements of the plurality of second structured elements have the same size.

### Embodiment 25

An article according to any one of embodiments 1 to 23, wherein two or more of the second structured elements of the plurality of second structured elements have different shapes.

### Embodiment 26

An article according to any one of embodiments 1 to 25, wherein the plurality of first structured elements are arranged in a uniformly spaced array.

### Embodiment 27

An article according to any one of embodiments 1 to 26, wherein the plurality of second structured elements are arranged in a uniformly spaced array.

### Embodiment 28

An article according to any one of embodiments 1 to 27, wherein each first structured element of the plurality of first structured elements comprises a maximum cross-sheet dimension of 2.5 centimeters or less when the malleable core is in a flat configuration.

### Embodiment 29

An article according to any one of embodiments 1 to 28, wherein each first structured element of the plurality of first structured elements comprises a maximum cross-sheet dimension of 1 centimeter or less when the malleable core is in a flat configuration.

### Embodiment 30

An article according to any one of embodiments 1 to 29, wherein each second structured element of the plurality of second structured elements comprises a maximum cross-sheet dimension of 2.5 centimeters or less when the malleable core is in a flat configuration.

### Embodiment 31

An article according to any one of embodiments 1 to 30, wherein each second structured element of the plurality of second structured elements comprises a maximum cross-sheet dimension of 1 centimeter or less when the malleable core is in a flat configuration.

### Embodiment 32

An article according to any one of embodiments 1 to 31, wherein the malleable core comprises metal.

### Embodiment 33

An article according to any one of embodiments 1 to 32, wherein the malleable core comprises at least one layer of metal foil.

### Embodiment 34

An article according to any one of embodiments 1 to 33, wherein the protrusion of each first structured element of the plurality of first structured elements comprises a circular shape.

### Embodiment 35

An article according to any one of embodiments 1 to 33, wherein the protrusion of each first structured element of the plurality of first structured elements comprises a non-circular shape.

### Embodiment 36

An article according to any one of embodiments 1 to 33, wherein the protrusion of each first structured element of the plurality of first structured elements comprises a polygonal shape.

### Embodiment 37

An article according to any one of embodiments 1 to 36, wherein each first structured element of the plurality of first structured elements comprises a compressible layer distal from the malleable core.

### Embodiment 38

An article according to embodiment 37, wherein the compressible layer on each first structured element of the plurality of first structured elements is not connected to the compressible layer on any adjacent first structured elements of the plurality of first structured elements.

### Embodiment 39

An article according to any one of embodiments 37 to 38, wherein the compressible layer exhibits compression set of 50% or less of an original thickness when tested according to ASTM D3575.

### Embodiment 40

An article according to any one of embodiments 1 to 39, wherein each second structured element of the plurality of second structured elements comprises a compressible layer distal from the malleable core.

### Embodiment 41

An article according to embodiment 40, wherein the compressible layer on each second structured element of the plurality of second structured elements is not connected to the compressible layer on any adjacent second structured elements of the plurality of second structured elements.

### Embodiment 42

An article according to any one of embodiments 40 to 41, wherein the compressible layer exhibits compression set of 50% or less of an original thickness when tested according to ASTM D3575.

### Embodiment 43

An article according to any one of embodiments 1 to 42, wherein the first coversheet and the second coversheet comprise a monolithic border about the perimeter of the shapeable member.

### Embodiment 44

An article according to any one of embodiments 1 to 42, wherein the first coversheet is attached to the second coversheet about a perimeter of the shapeable member.

### Embodiment 45

An article according to embodiment 44, wherein the first coversheet and the second coversheet are adhesively attached to each other about the perimeter of the shapeable member.

### Embodiment 46

An article according to embodiment 44, wherein the first coversheet and the second coversheet are chemically or thermally welded to each other about the perimeter of the shapeable member.

### Embodiment 47

An article according to any one of embodiments 1 to 46, wherein the first coversheet is attached to less than all of the first structured elements of the plurality of first structured elements.

### Embodiment 48

An article according to any one of embodiments 1 to 46, wherein the first coversheet is attached to all of the first structured elements of the plurality of first structured elements.

### Embodiment 49

An article according to any one of embodiments 47 to 48, wherein the first coversheet is adhesively attached to the first structured elements.

### Embodiment 50

An article according to any one of embodiments 47 to 48, wherein the first coversheet is chemically or thermally welded to the first structured elements.

### Embodiment 51

An article according to any one of embodiments 1 to 50, wherein the second coversheet is attached to less than all of the second structured elements of the plurality of second structured elements.

### Embodiment 52

An article according to any one of embodiments 1 to 50, wherein the second coversheet is attached to all of the second structured elements of the plurality of second structured elements.

### Embodiment 53

An article according to any one of embodiments 51 to 52, wherein the second coversheet is adhesively attached to the second structured elements.

### Embodiment 54

An article according to any one of embodiments 51 to 52, wherein the second coversheet is chemically or thermally welded to the second structured elements.

### Embodiment 55

An article according to any one of embodiments 1 to 54, wherein the first coversheet exhibits tensile elongation greater than zero and less than 1000%.

### Embodiment 56

An article according to any one of embodiments 1 to 55, wherein the first coversheet comprises one or more layers selected from a foam layer, a polymeric film, a nonwoven sheet, a woven sheet, a knitted sheet, a mesh sheet, and a net sheet.

### Embodiment 57

An article according to any one of embodiments 1 to 56, wherein the first coversheet comprises a compressible layer.

### Embodiment 58

An article according to any one of embodiments 56 to 57, wherein the compressible layer of the first cover sheet exhibits compression set of 50% or less of an original thickness when tested according to ASTM D3575.

### Embodiment 59

An article according to any one of embodiments 1 to 58, wherein the second coversheet exhibits tensile elongation greater than zero and less than 1000%.

### Embodiment 60

An article according to any one of embodiments 1 to 59, wherein the second coversheet comprises one or more layers selected from a foam layer, a polymeric film, a nonwoven sheet, a woven sheet, a knitted sheet, a mesh sheet, and a net sheet.

### Embodiment 61

An article according to any one of embodiments 1 to 60, wherein the second coversheet comprises a compressible layer.

### Embodiment 62

An article according to any one of embodiments 60 to 61, wherein the compressible layer of the second coversheet exhibits compression set of 50% or less of an original thickness when tested according to ASTM D3575.

### Embodiment 63

An article according to any one of embodiments 1 to 62, wherein the article comprises a perimeter in the general shape of a rectangle, with one or more tabs extending outward from at least one side of the rectangular, wherein each tab of the one or more tabs occupies less than all of the side from which it extends.

### Embodiment 64

An article according to embodiment 63, wherein the shapeable member extends into the area defined the one or more tabs.

### Embodiment 65

An article according to any one of embodiments 1 to 62, wherein the article comprises non-rectangular shape comprising a central portion and two or more fingers extending outwardly from the central portion, wherein the shapeable member extends into each finger of the two or more fingers.

### Embodiment 66

An article according to any one of embodiments 1 to 62, wherein the first coversheet and the second coversheet define an article perimeter having an article shape, and wherein the shapeable member located between the first coversheet and the second coversheet comprises a member perimeter defining a member shape that is different than the article shape.

### Embodiment 67

An article according to any one of embodiments 1 to 62, wherein the first coversheet and the second coversheet define an article perimeter having an article shape, and wherein the shapeable member located between the first coversheet and the second coversheet comprises a member perimeter defining a member shape that is the same as the article shape.

### Embodiment 68

An article according to any one of embodiments 1 to 67, wherein the shapeable member comprises one or more slits formed through the shapeable member, wherein each slit of the one or more slits extends from an edge of the shapeable member inward toward a center of the shapeable member.

### Embodiment 69

An article according to any one of embodiments 1 to 62, wherein the first coversheet and the second coversheet define an article perimeter having a generally rectangular shape, and wherein the shapeable member located between the first coversheet and the second coversheet comprises a member perimeter defining a non-rectangular member shape that is different than the generally rectangular shape of the article perimeter.

### Embodiment 70

An article according to embodiment 69, wherein the non-rectangular member shape comprises a generally rectangular shape comprising one or more tabs extending outward from at least one side of the rectangular, wherein each tab of the one or more tabs occupies less than all of the side from which it extends.

### Embodiment 71

An article according to any one of embodiments 1 to 70, wherein the shapeable member comprises a window opening formed therein, wherein the shapeable member forms a frame around the window opening.

### Embodiment 72

An article according to embodiment 71, wherein the first coversheet and the second coversheet extend over the window opening formed in the shapeable member.

### Embodiment 73

An article according to any one of embodiments 1 to 72, wherein the article comprises a stiffness of 100 N or less according to a three-bend test.

### Embodiment 74

An article according to any one of embodiments 1 to 73, wherein the first coversheet comprises an outer surface facing away from the shapeable member, and wherein the outer surface comprises a copolymer composition having a mean coefficient of friction of at least 0.2.

### Embodiment 75

An article according to any one of embodiments 1 to 73, wherein the first coversheet comprises an outer surface facing away from the malleable sheet, and wherein the outer surface comprises a copolymer composition having a mean coefficient of friction of up to 0.45.

### Embodiment 76

An article according to embodiment 75, wherein the mean coefficient of friction of the copolymer composition is at least 0.2.

### Embodiment 77

An article according to any one of embodiments 74 to 76, wherein the mean coefficient of friction of the copolymer composition is up to 0.35.

### Embodiment 78

An article according to any one of embodiments 1 to 77, wherein the second coversheet comprises an outer surface facing away from the malleable sheet, and wherein the outer surface of the second coversheet comprises a copolymer composition having a mean coefficient of friction of at least 0.2.

### Embodiment 79

An article according to any one of embodiments 1 to 77, wherein the second coversheet comprises an outer surface facing away from the malleable sheet, and wherein the outer surface of the second coversheet comprises a copolymer composition having a mean coefficient of friction of up to 0.45.

### Embodiment 80

An article according to embodiment 79, wherein the mean coefficient of friction of the copolymer composition on the outer surface of the second coversheet is at least 0.2.

### Embodiment 81

An article according to any one of embodiments 78 to 80, wherein the mean coefficient of friction of the copolymer composition on the outer surface of the second coversheet is up to 0.35.

### Embodiment 82

A manually shapeable surgical retractor comprising: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the surgical retractor comprises a perimeter in the general shape of a rectangle, with one or more tabs extending outward from at least one side of the rectangular, wherein each tab of the one or more tabs occupies less than all of the side from which it extends.

### Embodiment 83

A surgical retractor according to embodiment 82, wherein the shapeable member extends into the area defined the one or more tabs.

### Embodiment 84

A manually shapeable surgical retractor comprising: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the surgical retractor comprises non-rectangular shape comprising a central portion and two or more fingers extending outwardly from the central portion, wherein the shapeable member extends into each finger of the two or more fingers.

### Embodiment 85

A manually shapeable surgical retractor comprising: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the first coversheet and the second coversheet define a surgical retractor perimeter having a retractor shape, and wherein the shapeable member located between the first coversheet and the second coversheet comprises a member perimeter defining a member shape that is different than the retractor shape.

### Embodiment 86

A manually shapeable surgical retractor comprising: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the first coversheet and the second coversheet define a surgical retractor perimeter having a retractor shape, and wherein the shapeable member located between the first coversheet and the second coversheet comprises a member perimeter defining a member shape that is the same as the retractor shape.

### Embodiment 87

A manually shapeable surgical retractor comprising: a shapeable member comprising a malleable core, wherein the shapeable member comprises a first major surface and a second major surface, wherein the first major surface and the second major surface are on opposite sides of the shapeable member and the malleable core; a first coversheet attached to a first major surface of the shapeable member; and a second coversheet attached to a second major surface of the shapeable member; wherein the first coversheet and the second coversheet define an article perimeter having a generally rectangular shape, and wherein the shapeable member located between the first coversheet and the second coversheet comprises a member perimeter defining a non-rectangular member shape that is different than the generally rectangular shape of the article perimeter.

### Embodiment 88

A surgical retractor according to embodiment 87, wherein the non-rectangular member shape comprises a generally rectangular shape comprising one or more tabs extending outward from at least one side of the rectangular, wherein each tab of the one or more tabs occupies less than all of the side from which it extends.

### Embodiment 89

A surgical retractor according to any one of embodiments 82 to 88, wherein the shapeable member comprises one or more slits formed through the shapeable member, wherein each slit of the one or more slits extends from an edge of the shapeable member inward toward a center of the shapeable member.

### Embodiment 90

A surgical retractor according to any one of embodiments 82 to 89, wherein the first coversheet comprises an outer surface facing away from the malleable core, and wherein the outer surface of the first coversheet comprises a copolymer composition having a mean coefficient of friction of 0.2 to 0.45.

### Embodiment 91

A surgical retractor according to embodiment 90, wherein the coefficient of friction of the copolymer composition is up to 0.35.

### Embodiment 92

A surgical retractor according to any one of embodiments 90 to 91, wherein the second coversheet comprises an outer surface facing away from the malleable core, and wherein the outer surface of the second coversheet comprises a copolymer composition having a coefficient of friction of at least 0.2.

### Embodiment 93

A surgical retractor according to any one of embodiments 90 to 92, wherein the second coversheet comprises an outer surface facing away from the malleable core, and wherein the outer surface of the second coversheet comprises a copolymer composition having a coefficient of friction of up to 0.45.

### Embodiment 94

A surgical retractor according to embodiment 93, wherein the coefficient of friction of the copolymer composition on the outer surface of the second coversheet is at least 0.2.

### Embodiment 95

A surgical retractor according to any one of embodiments 92 to 94, wherein the coefficient of friction of the copolymer composition on the outer surface of the second coversheet is up to 0.35.

### Embodiment 96

A surgical retractor according to any one of embodiments 90 to 95, wherein the shapeable member comprises a stiffness of 100 N or less according to a three-bend test.

### Embodiment 97

A surgical retractor according to any one of embodiments 90 to 96, wherein the shapeable member comprises: a plurality of first structured elements attached to a first major surface of the malleable core, wherein each first structured element of the plurality of first structured elements comprises a protrusion extending away from the malleable core, wherein the plurality of first structured elements are spaced apart from each other by an inter-element distance when the malleable core is in a flat configuration, and wherein the inter-element distance between the plurality of first structured elements changes when the malleable core is deformed into a non-flat configuration; and optionally, a plurality of second structured elements attached to a second major surface of the malleable core, wherein each second structured element of the plurality of second structured elements comprises a protrusion extending away from the malleable core, wherein the plurality of second structured elements are spaced apart from each other by an inter-element distance when the malleable core is in a flat configuration, and wherein the inter-element distance between the plurality of second structured elements changes when the malleable core is deformed into a non-flat configuration.

### Embodiment 98

A surgical retractor according to any one of embodiments 90 to 97, wherein the malleable core comprises a metal foil layer.

### Embodiment 99

A surgical retractor according to any one of embodiments 97 to 98, wherein each first structured element of the plurality of first structured elements comprises a compressible layer distal from the malleable core.

### Embodiment 100

A surgical retractor according to embodiment 99, wherein the compressible layer on each first structured element of the plurality of first structured elements is not connected to the compressible layer on any adjacent first structured elements of the plurality of first structured elements.

### Embodiment 101

A surgical retractor according to any one of embodiments 97 to 100, wherein each second structured element of the plurality of second structured elements comprises a compressible layer distal from the malleable core.

### Embodiment 102

A surgical retractor according to embodiment 101, wherein the compressible layer on each second structured element of the plurality of second structured elements is not connected to the compressible layer on any adjacent second structured elements of the plurality of second structured elements.

### Embodiment 103

A surgical retractor according to any one of embodiments 90 to 102, wherein the first coversheet exhibits tensile elongation greater than zero and less than 1000%.

### Embodiment 104

A surgical retractor according to any one of embodiments 90 to 103, wherein the second coversheet exhibits tensile elongation greater than zero and less than 1000%.

### Embodiment 105

A shapeable article according to any one of embodiments 1 to 81 or a surgical retractor according to any one of embodiments 82 to 104, wherein the shapeable member comprises a member perimeter, and wherein an edge protector extends about at least a portion of the member perimeter, and wherein at least a portion of the edge protector is located between the first cover sheet and the second cover sheet.

### Embodiment 106

A shapeable article or a surgical retractor according to embodiment 105, wherein the edge protector comprises a compressible edge protector.

### Embodiment 107

A shapeable article or a surgical retractor according to any one of embodiments 105 to 106, wherein the edge protector extends about only a portion of the member perimeter of the shapeable member.

### Embodiment 108

A shapeable article or a surgical retractor according to any one of embodiments 105 to 106, wherein the edge protector extends about the entire member perimeter of the shapeable member.

### Embodiment 109

A kit comprising a lap sponge, a surgical retractor comprising the manually shapeable article according to any one of embodiments 1 to 81 or 105 to 108 or a surgical retractor according to any one of embodiments 82 to 108, and a frangible sealed package containing the lap sponge and the surgical retractor.

### Embodiment 110

A method of manufacturing a manually shapeable article according to any one of embodiments 1 to 81 and 105 to 108 or a surgical retractor according to any one of embodiments 82 to 108.

It should be understood that although exemplary articles, kits, and methods are described herein as “comprising” one or more components, features or steps, the methods may “comprise,” “consists of,” or “consist essentially of any of the above-described components and/or features and/or steps. Consequently, where the present invention, or a portion thereof, has been described with an open-ended term such as “comprising,” it should be readily understood that (unless otherwise stated) the description of the present invention, or the portion thereof, should also be interpreted to describe the present invention, or a portion thereof, using the terms “consisting essentially of or “consisting of or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, an article, kit, or method that “comprises” a list of elements (e.g., components or features or steps) is not necessarily limited to only those elements (or components or features or steps), but may include other elements (or components or features or steps) not expressly listed or inherent to the method.

As used herein, the singular forms “a,” “an,” and “the” include plural referents unless the context clearly dictates otherwise. Thus, for example, reference to “a” or “the” component may include one or more of the components and equivalents thereof known to those skilled in the art. Further, the term “and/or” means one or all of the listed elements or a combination of any two or more of the listed elements.

Further, the term “comprises” and variations thereof do not have a limiting meaning where these terms appear in the accompanying description. Moreover, “a,” “an,” “the,” “at least one,” and “one or more” are used interchangeably herein.

As used herein, the transitional phrases “consists of and “consisting of exclude any element, step, or component not specified. For example, “consists of or “consisting of used in a claim would limit the claim to the components, materials or steps specifically recited in the claim except for impurities ordinarily associated therewith (i.e., impurities within a given component). When the phrase “consists of or “consisting of appears in a clause of the body of a claim, rather than immediately following the preamble, the phrase “consists of or “consisting of limits only the elements (or components or steps) set forth in that clause; other elements (or components) are not excluded from the claim as a whole.

As used herein, the transitional phrases “consists essentially of and “consisting essentially of are used to define an article, kit, or method that includes materials, steps, features, components, or elements, in addition to those literally disclosed, provided that these additional materials, steps, features, components, or elements do not materially affect the basic and novel characteristic(s) of the claimed invention. The term “consisting essentially of occupies a middle ground between “comprising” and “consisting of. Further, it should be understood that the herein-described articles, kits, or methods may comprise, consist essentially of, or consist of any of the herein-described components and features, as shown in the figures with or without any additional feature(s) not shown in the figures. In other words, in some embodiments, the articles, kits, or methods of the present invention may have any additional feature that is not specifically shown in the figures. In some embodiments, the articles, kits, or methods of the present invention do not have any additional features other than those (i.e., some or all) shown in the figures, and such additional features, not shown in the figures, are specifically excluded from the articles, kits, or methods.

The complete disclosure of the patents, patent documents, and publications identified herein are incorporated by reference in their entirety as if each were individually incorporated. To the extent there is a conflict or discrepancy between this document and the disclosure in any such incorporated document, this document will control.

From the above disclosure of the general principles of the present invention and the preceding detailed description, those skilled in this art will readily comprehend the various modifications, re-arrangements and substitutions to which the present invention is susceptible, as well as the various advantages and benefits the present invention may provide. Therefore, the scope of the invention should be limited only by the following claims and equivalents thereof. In addition, it is understood to be within the scope of the present invention that the disclosed and claimed articles and methods may be useful in applications other than surgical procedures. Therefore, the scope of the invention may be broadened to include the use of the claimed and disclosed methods for such other applications.

1. A manually shapeable article comprising:
a shapeable member comprising:
a malleable core comprising a first major surface and a second major surface, wherein the first major surface and the second major surface are on opposite sides of the malleable core;
a plurality of first structured elements attached to the first major surface of the malleable core, wherein each first structured element of the plurality of first structured elements comprises a protrusion extending away from the malleable core, wherein the plurality of first structured elements are spaced apart from each other by an inter-element distance when the malleable core is in a flat configuration, and wherein the inter-element distance between the plurality of first structured elements changes when the malleable core is deformed into a non-flat configuration;
a plurality of second structured elements attached to the second major surface of the malleable core, wherein each second structured element of the plurality of second structured elements comprises a protrusion extending away from the malleable core, wherein the plurality of second structured elements are spaced apart from each other by an inter-element distance when the malleable core is in a flat configuration, and wherein the inter-element distance between the plurality of second structured elements changes when the malleable core is deformed into a non-flat configuration;
a first coversheet attached to the shapeable member, wherein the first coversheet covers the plurality of first structured elements of the shapeable member; and
a second coversheet attached to the shapeable member, wherein the second coversheet covers the plurality of second structured elements of the shapeable member, such that the malleable core is located between first and second coversheets.

2. An article according to claim 1, wherein the plurality of first structured elements are attached to each other by a land portion extending between the plurality of first structured elements.

3. An article according to claim 1, wherein the malleable core comprises metal.

4. An article according to claim 1, wherein the malleable core comprises at least one layer of metal foil.

5. An article according to claim 1, wherein one or both of (a) each first structured element of the plurality of first structured elements and (b) each second structured element of the plurality of second structured elements comprises a compressible layer distal from the malleable core.

6. An article according to claim 5, wherein the compressible layer on one or both of (a) each first structured element of the plurality of first structured elements and (b) each second structured element of the plurality of second structured elements is not connected to the compressible layer on any adjacent first structured elements of the plurality of first structured elements.

7. An article according to claim 5, wherein the compressible layer exhibits compression set of 50% or less of an original thickness when tested according to ASTM D3575.

8. An article according to claim 1, wherein one or both of the first coversheet and second coversheet exhibits tensile elongation greater than zero and less than 1000%.

9. An article according to claim 1, wherein one or both of the first coversheet and second coversheet comprises one or more layers selected from a foam layer, a polymeric film, a nonwoven sheet, a woven sheet, a knitted sheet, a mesh sheet, and a net sheet.

10. An article according to claim 1, wherein one or both of the first coversheet and second coversheet comprises a compressible layer.

11. An article according to claim 10, wherein the compressible layer of one or both of the first cover sheet and second coversheeet exhibits compression set of 50% or less of an original thickness when tested according to ASTM D3575.

12. An article according to claim 1, wherein the shapeable member comprises a window opening formed therein, wherein the shapeable member forms a frame around the window opening.

13. An article according to claim 1, wherein the article comprises a stiffness of 100 N or less according to a three-bend test.

14. An article according to claim 1, wherein one or both of the first coversheet and second coversheet comprises an outer surface facing away from the shapeable member, and wherein the outer surface comprises a copolymer composition having a mean coefficient of friction of at least 0.2.

15. An article according to claim 1, wherein one or both of the first coversheet and second coversheet comprises an outer surface facing away from the malleable sheet, and wherein the outer surface comprises a copolymer composition having a mean coefficient of friction of up to 0.45.

16. A manually shapeable surgical retractor comprising:
a shapeable member comprising a malleable core, wherein the shapeable member comprises a first major surface and a second major surface, wherein the first major surface and the second major surface are on opposite sides of the shapeable member and the malleable core;
a first coversheet attached to a first major surface of the shapeable member; and
a second coversheet attached to a second major surface of the shapeable member;
wherein the surgical retractor comprises a perimeter in the general shape of a rectangle, with one or more tabs extending outward from at least one side of the rectangular, wherein each tab of the one or more tabs occupies less than all of the side from which it extends.

17. A shapeable article according to claim 1 or a surgical retractor according to claim 16, wherein the shapeable member comprises a member perimeter, and wherein an edge protector extends about at least a portion of the member perimeter, and wherein at least a portion of the edge protector is located between the first cover sheet and the second cover sheet.

18. A shapeable article or a surgical retractor according to claim 17, wherein the edge protector comprises a compressible edge protector.

19. A shapeable article or a surgical retractor according to claim 17, wherein the edge protector extends about only a portion of the member perimeter of the shapeable member.

20. A shapeable article or a surgical retractor according to claim 17, wherein the edge protector extends about the entire member perimeter of the shapeable member.
